(19) United States
(12) Patent Application Publication
(10) Pub. No.: US 2019/0259469 A1
(43) Pub. Date: Aug. 22, 2019

(54) Method for Evaluating Genotoxicity of Substance

(71) Applicant: Kao Corporation, Tokyo (JP)

(72) Inventors: Shoji Matsumura, Saitama-shi (JP); Hiroshi Honda, Utsunomiya-shi (JP)

(73) Assignee: Kao Corporation, Tokyo (JP)

(21) Appl. No.: 16/269,980

(22) Filed: Feb. 7, 2019

Related U.S. Application Data
(63) Continuation of application No. PCT/JP2017/005700, filed on Feb. 16, 2017.

(51) Int. Cl. G16B 20/50; C12Q 1/6886; G16B 20/20

(52) U.S. Cl. CPC G16B 20/50; C12Q 1/6886; C12Q 2600/166; C12Q 2600/156; G16B 20/20

(57) ABSTRACT

It is intended to provide a method for conveniently analyzing mutations in cells at low cost. The present invention provides a method for analyzing mutations in a cell population, comprising: obtaining DNAs derived from the cell population; sequencing fragments of the DNAs to obtain one or more read sequences per fragment; comparing each of the one or more read sequences with a reference sequence to detect sites of mismatch bases between the each read sequence and the reference sequence; obtaining the sites thus detected in the one or more read sequences as sites of mutation; and obtaining information on mutations at the sites of mutation and analyzing the tendencies of the mutations on the basis of the information.

### REFERENCE TO SEQUENCE LISTING SUBMITTED ELECTRONICALLY

The content of the electronically submitted substitute sequence listing, file name 2537_1690001_SL_ST25.txt, size 6,316,495 bytes; and date of creation Feb. 7, 2019, filed herewith, is incorporated herein by reference in its entirety.

### FIELD OF THE INVENTION

The present invention relates to a method for analyzing mutations or evaluating the genotoxicity of a substance.

### BACKGROUND OF THE INVENTION

Genotoxicity is a generic term for toxicities to intracellular genetic materials which is mainly DNA and refers to, in a narrower sense, the property of causing damage, mutation and others in DNA to alter its genetic information (mutagenicity). The genetic information in DNA is retained by a nucleotide sequence constituted by 4 bases of A (adenine), T (thymine), G (guanine) and C (cytosine). A genotoxic substance acts directly or indirectly on DNA and causes qualitative or quantitative change in its nucleotide sequence to alter its genetic information. Such alteration in genetic information by a genotoxic substance is known to cause carcinogenesis or reproductive and developmental toxicity. Thus, the evaluation of medicines, cosmetics, various chemical substances, etc. for their genotoxicity is important for public safety.

Diverse mechanisms underlie the genotoxicity and they are broadly divided into: a base pair substitution mutation which alters base pair information in DNA to a different base pair; a short insertion or deletion mutation which causes the insertion or deletion of a short nucleotide sequence in the sequence of DNA; and a genomic structural variation which changes a genomic structure by causing the insertion, deletion, translocation, inversion or the like of a relatively long nucleotide sequence in the whole genomic sequence. In particular, the short insertion or deletion mutation is also called frameshift mutation when this mutation changes the reading frame of a protein encoded by a gene. Reportedly, the genotoxicity of a chemical substance often causes a base pair substitution mutation or a short insertion or deletion mutation.

Various genotoxicity tests using in vitro or in vivo models have been developed so far as methods for evaluating the genotoxicity of a substance. For example, a test of detecting the base pair substitution mutation, or the frameshift mutation mentioned above includes an Ames test developed by Professor Bruce N. Ames (Non Patent Literature 1). The Ames test employs Salmonella strains which have a mutation in histidine biosynthetic gene and cannot survive in a medium free from histidine. When the Salmonella strains are exposed to a substance and thereby become capable of synthesizing histidine through a mutation in the gene, these strains become able to form colonies on the medium free from histidine. The mutagenicity of the substance is confirmed by counting the number of colonies formed. In addition, for example, a micronucleus test using mammalian cells is used as a test for detecting the presence or absence of a genomic structural variation (Non Patent Literature 2). Furthermore, the presence or absence of the genotoxicity of a substance can be confirmed highly sensitively by combining a plurality of genotoxicity tests.

However, the detection of genotoxicity in the conventional genotoxicity tests as mentioned above depends on an indirect indicator which does not directly reflect the quantity or quality of a mutation. Therefore, the conventional tests cannot gain detailed qualitative and quantitative mutation information such as the type of the mutation or one in every how many bases the mutation occurs. Since there is no unified indicator among various tests, it is difficult to compare results among different tests. Thus, the conventional genotoxicity tests do not provide sufficient information for pursuing the systemic understanding of genotoxicity, such as the comparison of strength among a plurality of mutagens or the classification of genotoxicity according to mechanisms.

The application of high-throughput sequencing technology using a next-generation sequencer or the like to genotoxicity evaluation has been proposed. Maslov et al. (Non Patent Literature 3) disclose a methodology for the application of high-throughput sequencing to genotoxicity evaluation. One of the methods involves exposing cells to a substance, then preparing a population with uniform genomic information derived from a single cell, and obtaining the genomic information using a next-generation sequencer to identify a mutation site. As practical use of this methodology, for example, Matsuda et al. (Non Patent Literature 4) have reported that individual mutations and their positions were identified by isolating a single colony of a Salmonella typhimurium-derived TA100 strain exposed to a mutagen, obtaining the whole genomic sequence thereof using a next-generation sequencer, and comparing read sequences with a reference sequence to detect, as a mutation site, a site having a base change commonly occurs among a plurality of read sequences with a given frequency. Matsuda et al. (Non Patent Literature 5) have further reported a method which involves harvesting a trace amount of a diluted strain culture solution instead of isolating a single colony, additionally culturing the strain, and detecting mutations by the same approach as in Non Patent Literature 4 using the resulting cultures. A method for evaluating the accumulation of DNA mutations by radiation or the like using a next-generation sequencer has been reported as another method.

Specifically, a sequence specific for a restriction site or the like (tag sequence) is focused on, and evaluation is conducted on the basis of the frequency in appearance of the sequence to predict a mutation frequency in the genome (Patent Literature 1). A method for detecting mutations is also disclosed which involves adding a unique tag sequence to each molecule of cell-free (cf) DNA, obtaining consensus sequences of a plurality of read sequences from the same molecule, then aligning the plurality of read sequences at the same location on the genome and comparing them (Non Patent Literatures 6 and 7).
(Patent Literature 1) WO 2014/175427
(Non Patent Literature 1) Mortelmans et al., Mutation Research, 2000, 455: 29-60
(Non Patent Literature 2) Matsushima et al., Mutagenesis, 1999, 14: 569-580
(Non Patent Literature 3) Maslov et al., Mutation Research 2015, 776: 136-143
(Non Patent Literature 4) Matsuda, Genes and Environment, 2013, 35: 53-56
(Non Patent Literature 5) Matsuda et al., Genes and Environment, 2015, 37: 15-24
(Non Patent Literature 6) Nucleic Acids Research, 2016, 44 (11): e105
(Non Patent Literature 7) Clinical Oncology, 2016, 28: 735-738

### SUMMARY OF THE INVENTION

In one embodiment, the present invention provides a method for evaluating the genotoxicity of a test substance, comprising:

(1) obtaining DNAs from a test group, the test group is a cell population exposed to the test substance;
(2) sequencing fragments of the DNAs to obtain one or more read sequences per fragment;
(3) comparing each of the one or more read sequences with a reference sequence to detect sites of mismatch bases between the each read sequence and the reference sequence, wherein the reference sequence is a known sequence in the DNAs;
(4) obtaining the sites detected in the step (3) as sites of mutation each having a base pair substitution mutation;
(5) classifying each of the obtained mutations according to base pair mutation patterns; and
(6) determining respective mutation frequencies of the mutation patterns obtained in the step (5).

In an alternative embodiment, the present invention provides a method for evaluating the genotoxicity of a test substance, comprising:

(1′) obtaining DNAs from a test group, the test group is a cell population exposed to the test substance;
(2′) sequencing fragments of the DNAs to obtain one or more read sequences per fragment;
(3′) comparing each of the one or more read sequences with a reference sequence to detect sites of mismatch bases between the each read sequence and the reference sequence, wherein the reference sequence is a known sequence in the DNAs;
(4′) obtaining the sites detected in the step (3′) as sites of mutation each having a base pair substitution mutation;
(5′) as to each of the mutations thus obtained, determining a context sequence comprising a base before mutation and adjacent upstream and downstream bases of the base before mutation, the context sequence is determined on the basis of the reference sequence;
(6′) typing each of the mutations obtained in the step (4′) according to the context sequence determined in the step (5′) and the type of the base after mutation; and
(7′) determining respective mutation frequencies of the mutation types obtained in the step (6′).

In a further alternative embodiment, the present invention provides a method for evaluating the genotoxicity of a test substance, comprising:

(1″) obtaining DNAs from a test group, the test group is a cell population exposed to the test substance;
(2″) sequencing fragments of the DNAs to obtain one or more read sequences per fragment;
(3″) comparing each of the one or more read sequences with a reference sequence to detect sites of base insertion or deletion in the each read sequence with respect to the reference sequence, wherein the reference sequence is a known sequence in the DNAs;
(4″) obtaining the sites detected in the step (3″) as sites of mutation each having an insertion or deletion mutation;
(5″) as to each of the mutations thus obtained, determining the base length of the insertion or deletion and/or the type of the inserted base; and
(6″) determining respective mutation frequencies for each of the sites of the insertion or deletion mutations with different base lengths of the insertion or deletion and/or different types of the inserted bases determined in the step (5″).

In a further alternative embodiment, the present invention provides a method for evaluating mutations in cancer cells, comprising:

(1) obtaining DNAs from a test group, the test group is a cancer cell population;
(2) sequencing fragments of the DNAs to obtain one or more read sequences per fragment;
(3) comparing each of the one or more read sequences with a reference sequence to detect sites of mismatch bases between the each read sequence and the reference sequence, wherein the reference sequence is a known sequence in the DNAs;
(4) obtaining the sites detected in the step (3) as sites of mutation each having a base pair substitution mutation;
(5) classifying each of the obtained mutations according to base pair mutation patterns; and
(6) determining respective mutation frequencies of the mutation patterns obtained in the step (5).

In a further alternative embodiment, the present invention provides a method for evaluating genetic information in cultured cells, comprising:

(1) obtaining DNAs from a test group, the test group is a cultured cell population;
(2) sequencing fragments of the DNAs to obtain one or more read sequences per fragment;
(3) comparing each of the one or more read sequences with a reference sequence to detect sites of mismatch bases between the each read sequence and the reference sequence, wherein the reference sequence is a known sequence in the DNAs;
(4) obtaining the sites detected in the step (3) as sites of mutation each having a base pair substitution mutation;
(5) classifying each of the obtained mutations according to base pair mutation patterns; and
(6) determining respective mutation frequencies of the mutation patterns obtained in the step (5).

### BRIEF DESCRIPTION OF THE DRAWINGS

FIG. 1A and FIG. 1B show the mutation call ratios of base pair mutation patterns in synthetic DNA samples. FIG. 1A: GC base pair mutation patterns, FIG. 1B: AT base pair mutation patterns.

FIG. 2A and FIG. 2B show the increased mutation frequencies of base pair mutation patterns in synthetic DNA samples. FIG. 2A: GC base pairs, FIG. 2B: AT base pairs.

FIG. 3A and FIG. 3B show the increased mutation frequencies of insertion mutations in synthetic DNA samples. FIG. 3A: the numbers of inserted bases, FIG. 3B: the types of inserted bases.

FIG. 4A and FIG. 4B show the increased mutation frequencies of base pair mutation patterns in samples exposed to a mutagen. FIG. 4A: GC base pairs, FIG. 4B: AT base pairs. *: p<0.05, **: p<0.01, ***: p<0.001 (Dunnett's test).

FIG. 5 shows the difference, depending on the types of original bases, in the increased mutation frequencies of base pair mutation patterns in samples exposed to a mutagen.

FIG. 6 shows spectral analysis results of base pair mutation patterns in samples exposed to a mutagen.

FIG. 7 shows sequence context analysis results of base pair mutations in samples exposed to a mutagen. Signature 11: patterns of mutation signatures obtained using a known alkylating agent, ENU: mutation patterns obtained by ethylnitrosourea treatment in Example 2.

FIG. 8 shows sequence context analysis results of base pair mutations in samples exposed to a mutagen (continued from FIG. 7). Signature 11: patterns of mutation signatures obtained using a known alkylating agent, ENU: mutation patterns obtained by ethylnitrosourea treatment in Example 2.

FIG. 9, “Schematic diagram 1,” shows a schematic diagram of the procedures for preparing the DNA sample.

FIG. 10A-10D, “Schematic diagram 2,” shows a schematic diagram of the four steps in the editing and analysis flows of the read sequence. FIG. 10A shows step 1: the merging of pair reads. FIG. 10B shows step 2: mapping onto the reference sequence. FIG. 10C shows step 3: the restriction to the marged regions of reads on the basis of quality values. FIG. 10D shows step 4: the direct extraction of the mutation site from the read.

FIG. 11, “Schematic diagram 3,” shows a schematic diagram of the mutation analysis algorithm for base pair substitution.

### DETAILED DESCRIPTION OF THE INVENTION

### 1. Definition

In the present specification, the term “mutation” refers to a mutation in DNA. Examples thereof include the deletion, insertion, substitution, addition, inversion, and translocation of a base or a sequence in DNA. In the present specification, the mutation encompasses the deletion, insertion, substitution, and addition of one base, and the deletion, insertion, substitution, addition, inversion, and translocation of a sequence consisting of two or more bases. In the present specification, the mutation also includes mutations in a coding region and a noncoding region and also includes a mutation which changes an expressed amino acid and a mutation which does not change an expressed amino acid (silent mutation).

In the present invention, the “genotoxicity” of a substance to be evaluated refers to the property of causing a mutation by the substance (so-called mutagenicity).

In the present specification, the term “original fragment” is a fragment of DNA to be analyzed and refers to a single-stranded DNA fragment whose sequence is to be read through sequencing reaction. In the present specification, the term “original base” as to a base at a mutation site refers to a base before the mutation at a location of the mutation in the original fragment.

All patent literatures, non patent literatures, and other publications cited herein are incorporated herein by reference in their entirety.

### 2. Method for Analyzing Mutation in Cell Population

A method for evaluating genotoxicity by use of high-throughput sequencing is expected to be able to directly evaluate the quantity or quality of a mutation caused by a mutagen. Also, such a method is considered basically applicable to every organism species as long as a genomic sequence thereof is available. Meanwhile, a conventional method for evaluating genotoxicity by use of high-throughput sequencing, as disclosed in Non Patent Literature 4, adopts a method of aligning and comparing all of a large number of read sequences including a site of interest, in order to detect a base mutation at one site. Such a method therefore requires a great deal of sequence information and requires enormous time, labor and cost for the obtainment of the sequence information and the detection of the mutation site. Furthermore, a mutation induced by exposure to a substance generally has a very low frequency and does not evenly occur among individual cells within a population. Therefore, it is considered difficult to accurately evaluate a mutation frequency within a cell population or the influence of a mutagen on the cell population by the analysis of single cells as disclosed in Non Patent Literature 4. In the method described in Non Patent Literature 5, genetic information in samples subjected to analysis was still uniform. Therefore, the results did not reflect information in the cell population. For the evaluation of genotoxicity, important points are how to detect low-frequency mutations contained in individual cells and how to evaluate the genotoxicity to the population on the basis of the detection results.

If the analysis as disclosed in Non Patent Literature 4 or 5 is repetitively conducted for samples derived from a plurality of different single cells, information which reflects events in the cell population can be gained. However, time, labor and cost required therefor are enormous. This method is not realistic, particularly, for evaluating a plurality of substances or for evaluating a dose-response relationship. On the other hand, the method of Patent Literature 1, albeit with relatively low cost, merely predicts a mutation frequency through the use of a specific sequence such as a restriction site. This method has low qualitative and quantitative performance for mutations in the whole genome and is not capable of achieving genotoxicity evaluation based on accurate mutation information.

The conventional methods for evaluating genotoxicity by use of high-throughput sequencing are very difficult to apply to the evaluation of a plurality of substances, in consideration of technical hurdles for the isolation and culture of mammalian cells or increased mutation analysis cost attributed to genome size. There is a demand for the development of a convenient and low-cost method for detecting quantitative and qualitative information on mutations caused by a mutagen in a cell population retaining various pieces of mutation information.

The present inventors have found an analysis method which involves comparing each of read sequences with a reference sequence to detect a base mutation in each individual read sequence, and calculating the pattern and frequency of the mutation by the analysis of the results, instead of the conventional methods which involve comparing a plurality of read sequences containing a specific site of a reference sequence and thereby detecting, as a mutation site, a site where a nucleotide sequence at the specific site is altered with a given frequency in the plurality of read sequences. This analysis method may permit mutation detection based on a great deal of nucleotide sequence information, or high-throughput and highly sensitive mutation detection more efficiently than the conventional methods, and provide data which reflects the quantitative and qualitative tendencies of mutations as the whole cell population.

The method of the present invention is capable of obtaining quantitative and qualitative information on mutations within a cell population by single analysis. Thus, the method of the present invention permits mutation analysis at a cell population level much more conveniently and at much lower cost as compared with the conventional methods. The method of the present invention is particularly effective for analysis to determine tendencies of mutations in a cell population having heterogeneous genetic information, such as the genotoxicity evaluation of substances or the evaluation of cancer.

Thus, in one embodiment, the present invention provides a method for analyzing mutations in a cell population. The method of the present invention has the following basic procedures of:

(A) obtaining DNAs derived from the cell population;
(B) sequencing fragments of the DNAs (i.e., original fragments) to obtain one or more read sequences per fragment;
(C) comparing each of the one or more read sequences with a reference sequence to detect sites of mismatch bases between the each read sequence and the reference sequence;
(D) obtaining the sites thus detected in the one or more read sequences as mutation sites; and
(E) obtaining information on the mutations at the mutation sites and analyzing the tendencies of the mutations on the basis of the information.

The cell population used in the method of the present invention may be a homogeneous population (e.g., a cell population derived from a single colony, which has uniform genetic information), may be a heterogeneous population (population having non-uniform genetic information), or may be a population having unknown uniformity of genetic information. A heterogeneous population or a putative heterogeneous cell population is preferred. Examples of the cell population used in the method of the present invention include specimens harvested from animals or plants, and populations of animal-, plant- or microbe-derived cultured cells and preferably include populations of animal, plant or microbe strain-derived cultured cells. Examples of the animal preferably include mammals such as humans, silkworms, and nematodes. Examples of the microbes preferably include E. coli, Salmonella, and yeasts. Other examples of the cell population used in the method of the present invention include specimens harvested from living bodies and cultures thereof, cultured cells exposed to a mutagen or a candidate substance thereof, and cultured cells given a drug or a candidate substance thereof.

The cell population-derived DNAs used in the present invention can be obtained by extraction or isolation from the cell population by use of a usual method in the art. In the extraction or isolation, for example, a commercially available DNA extraction kit can be used. Alternatively, the cell population-derived DNAs preserved after the extraction or isolation may be obtained and used in the method of the present invention. Examples of the cell population-derived DNA used in the present invention include genomic DNA, mitochondrial genomic DNA, chloroplastic genomic DNA, plasmid DNA, and viral genomic DNA. Among them, genomic DNA is preferred.

Alternatively, in the case of analyzing RNA virus in the cell population, RNAs may be obtained and analyzed instead of the DNAs. The RNAs in cells can be extracted or isolated by a usual method in the art using a commercially available RNA extraction kit or the like. Alternatively, the cell population-derived RNAs preserved after the extraction or isolation may be obtained and used in the method of the present invention. In the case of obtaining and analyzing RNA in the method of the present invention, the “DNA” in the present specification is replaced with “RNA”, and the base T is replaced with the base U.

The reference sequence used in the method of the present invention is a known sequence contained in the DNAs which are subject to the mutation analysis. In this context, it is preferred to use a sequence registered in a public database or the like as the known sequence. Otherwise the known sequence that may be used is a sequence in the DNAs to be analyzed, sequenced in advance using a sequencer or the like prior to the step (C) in the method of the present invention. The reference sequence is not particularly limited by its region, length or the number thereof and can be appropriately selected from the DNAs according to the purpose of the mutation analysis. When the purpose of the mutation analysis according to the present invention is, for example, the genotoxicity evaluation of a substance, the length of the reference sequence is not particularly limited and is preferably 1,000 bp or more, more preferably 10,000 bp or more, even more preferably 100,000 bp or more, even more preferably 1,000,000 bp or more, in total.

The fragmentation of the DNAs can be conducted by use of a usual method in the art, such as ultrasonic treatment or enzymatic treatment. The length of each fragment to be prepared can be appropriately selected according to a length which can be accurately read by a sequencer. In general, 100 to 10,000 bp can be selected. However, a fragment having a length of 10,000 bp or more may be prepared as long as the length can be accurately read by a sequencer. A more appropriate range of the length can be selected depending on the type of the sequencer. For example, in the case of using a sequencer for sequencing reaction which includes a fragment amplification step, the length of the fragment is preferably 100 to 500 bp on average, more preferably 150 to 200 bp on average. For example, in the case of using a sequencer conducting single-molecule real-time sequencing, the length of the fragment is preferably 150 to 10,000 bp on average, more preferably 200 to 1,000 bp on average, even more preferably 200 to 500 bp on average.

Subsequently, the obtained fragments are sequenced. The sequencing of moieties to be used in sequence comparison with the reference sequence mentioned later suffices for the sequencing of the fragments. For example, fragments having a sequence in which at least a portion, preferably the whole corresponds to the DNA region of the reference sequence, can be sequenced. For mammalian cells or the like, exon regions or the like may be selectively sequenced. A kit for region selection such as SureSelect (manufactured by Agilent Technologies, Inc.) is available on the market.

In the sequencing reaction which includes a fragment amplification step, the fragments are amplified by PCR or the like while each amplified fragment is sequenced. In the single-molecule real-time sequencing reaction, the fragments are sequenced without amplification. In the sequencing of the fragments, a sequencer known in the art can be used. Preferably, a high-throughput sequencer (so-called next-generation sequencer) is used. HiSeq (manufactured by Illumina, Inc.), MiSeq (manufactured by Illumina, Inc.), or the like is available on the market as the high-throughput sequencer which conducts the fragment amplification. Also, Pac Bio RS II (manufactured by Pacific Biosciences of California, Inc.), Pac Bio Sequel System (manufactured by Pacific Biosciences of California, Inc.), or the like is available on the market as the high-throughput sequencer which conducts single-molecule real-time sequencing.

Detailed procedures for the method for sequencing the fragments are not particularly limited. A sequencing method with higher accuracy is preferred. One example of such a method for sequencing including a fragment amplification step includes a method of obtaining sequences from both ends of each fragment and exploiting a common moiety, as mentioned later. Comparison accuracy may be further improved by comparing complementary strands of sequences obtained from two original fragments complementary to each other. Examples of such a technique include a method of adding adaptors unique to each fragment molecule during HiSeq or MiSeq library preparation and referring to the nucleotide sequences of complementary strands on the basis of the adaptor sequence information after sequencing (Proc Natl Acad Sci USA, 2012, 109 (36): 14508-14513). Examples of such a method for single-molecule real-time sequencing include a method of adding adaptors having a hairpin sequence to both ends of each double-stranded fragment to prepare a one cyclic form, continuously sequencing several times a single molecule of the cyclic fragment, and integrating the obtained pieces of sequence information (Nucleic Acids Res, 2010, 38 (15): e159).

As a result of the sequencing described above, results (read sequences) of reading each of the original fragments obtained by the DNA fragmentation mentioned above are obtained. The number of read sequences obtained for each original fragment can be one or more. Preferably, a plurality of read sequences are obtained for each fragment from the viewpoint of improvement in the accuracy of analysis on mutation tendencies mentioned later. The number of read sequences obtained from one fragment is preferably 2 or more, more preferably 10 or more. On the other hand, the number of read sequences is preferably 5 or less, more preferably 2 or less, from the viewpoint of analysis efficiency.

The one or more read sequences thus obtained can be used directly in subsequent comparison with the reference sequence. It is preferred to extract bases with high reading reliability of the sequencing from among the bases of the obtained read sequences, from the viewpoint of improvement in the accuracy of the mutation analysis. Preferably, highly reliable bases are extracted from among the corresponding bases of two or more read sequences obtained from one fragment. In the present specification, the extracted highly reliable bases are referred to as “consensus bases”. The extraction of the “consensus bases” can be conducted using a program or the like attached to the next-generation sequencer. More specific examples of the procedures include: a method of extracting, as the “consensus bases”, bases of the same type at corresponding positions between two read sequences obtained from one fragment, or complementary bases at the positions when the two read sequences are complementary strands; a method of comparing bases between two or more read sequences obtained from one fragment, determining a base (including complementary bases in complementary strands) appearing with the largest frequency at each position on the sequences, and extracting these bases as the “consensus bases”; a method of acquiring, as the “consensus bases”, bases with the highest accuracy (quality value) in reading by a sequencer among the bases at corresponding positions of the read sequences; a method of probabilistically determining the “consensus bases” on the basis of quality values, base appearance frequencies, etc.; a method in which, when the corresponding bases are matched among all read sequences obtained from one fragment, the base is determined as the “consensus base”; and a combined method thereof. In the method of the present invention, the extraction of the “consensus bases” may be optionally carried out before or after mapping of the read sequences to the reference sequence mentioned later.

Subsequently, each of the one or more read sequences obtained by the sequencing from each original fragment is mapped to the reference sequence for sequence comparison. As a result of the comparison, mismatched sites of bases between each of the read sequences and the reference sequence are detected. Preferably, the comparison can be conducted only for the “consensus bases” mentioned above on the read sequences. Examples of the type of the “mismatched site” include a site where the type of a base on the read sequence differs from the reference sequence (substitution site), a site where a base on the read sequence is deleted with respect to the reference sequence (deletion site), and a site where a base is inserted on the read sequence with respect to the reference sequence (insertion site).

The detected “mismatched site” is obtained as a mutation site in the DNA to be analyzed. More specifically, information on a mutation at the mutation site is obtained. Examples of the information to be obtained include, but are not limited to, the type of the mismatched site (mutation site) (e.g., a substitution site, a deletion site or an insertion site), the type of the base at the site and the type of a base (or a base pair) before the mutation (e.g., the type of a base at a position corresponding to the site on the reference sequence), and the types of both adjacent bases of the site (e.g., the types of both bases adjacent to the position corresponding to the site on the reference sequence). In a preferred embodiment, when the mismatched site is a substitution site, the type of the base at the site and the type of a base before the mutation is obtained as the information; when the mismatched site is a deletion site, the type of a base before the mutation and the types of both adjacent bases of the deletion site are obtained as the information; and when the mismatched site is an insertion site, the type of the base at the site and the types of both adjacent bases of the insertion site are obtained as the information.

Sites of match bases between the read sequence and the reference sequence can also be detected from the comparison of the read sequence with the reference sequence. These “match sites” can be obtained as mutation-free sites in the DNA to be analyzed. Information on these mutation-free sites can be obtained. Examples of the information to be obtained include the types of the bases at the sites and the types of both adjacent bases of the sites.

Information on the mutation at the mutation site mentioned above, or information on the mutation-free sites is obtained as to each of the one or more read sequences. The obtained pieces of information can be collected to create a database for mutation analysis. For example, a database of all information on the mutation site obtained from each read sequence may be created; a database may be created in which mutation information obtained from each read sequence is classified by type of the mutation site; a database may be created in which mutation information obtained from each read sequence is classified by type of a base before the mutation (e.g., in the reference sequence) or after the mutation at the mutation site; a database may be created in which mutation information obtained from each read sequence is classified by base length of the mutation site (e.g., length of an insertion, deletion or substitution site); or a database may be created by combining these classifications. Alternatively, a database may be created in which information on the mutation site and information on the mutation-free sites are unified. For example, a database may be created in which the information on the mutation site and the information on the mutation-free sites are compiled by type of a base (A, T, G and C) before the mutation (e.g., in the reference sequence). Alternatively, a database may be created together with information on whether the identified position of the mutation site on the genome corresponds to a coding region or a noncoding region of a gene and, if the mutation site is in the coding region, information on whether the coding region is an intron or an exon or resides in a strand to be transcribed into RNA or not.

The detection of the “mismatched site” and the obtainment of information on the mutation site or the mutation-free sites mentioned above may be conducted for all the read sequences obtained by the sequencing, or may be conducted for part of read sequences. The total amount of the read sequences used in the detection (total length of the read sequences used) is not particularly limited as long as the amount permits subsequent analysis on the tendencies of mutations. The total amount of the read sequences used is preferably a base length equal to or more than the reciprocal of a mutation frequency, more preferably a base length that is 100 times or more of the reciprocal of a mutation frequency. For example, since a mutation frequency in Example 2 mentioned later is of an order of approximately 1/10⁵ bp, the total length of the read sequences used is preferably 1×10⁵ bp or more, more preferably 1×10⁷ bp or more, even more preferably 1×10⁹ bp or more. The total amount of the read sequences used in the detection is preferably 10 times the amount described above for analyzing the tendency of a mutation having a mutation frequency of an order of 1/10⁶ bp, and is preferably 1/10 times the amount described above for analyzing the tendency of a mutation having a mutation frequency of an order of 1/10⁴ bp. On the other hand, the total amount of the read sequences used in the detection is preferably a base length that is 10,000 times or less of the reciprocal of a mutation frequency, more preferably a base length that is 1,000 times or less of the reciprocal of a mutation frequency, even more preferably a base length that is 100 times or less of the reciprocal of a mutation frequency, from the viewpoint of analysis efficiency. For example, the total amount of the read sequences used in the detection is preferably 1×10¹⁰ bp or less, more preferably 1×10⁹ bp or less, even more preferably 1×10⁸ bp or less, even more preferably 1×10⁷ bp or less, even more preferably 1×10⁶ bp or less. The database for mutation analysis may be created on the basis of information on all mutation sites or mutation-free sites obtained or may be created on the basis of information only on part of sites as long as the database permits subsequent analysis on the tendencies of mutations.

In the conventional methods (e.g., Non Patent Literatures 4 and 5), a plurality of read sequences corresponding to a specific site of a reference sequence are obtained. Then, when the same type of a mismatched base is found with a given frequency at the same site among the plurality of read sequences, the site is determined as a mutation site in the DNA to be analyzed. This method might overlook a low-frequency mutation. Also, this method restricts a DNA region for mutation detection to a region having a limited length and having overlaps of the plurality of read sequences and requires a great deal of data for overlapping reads. Therefore, enormous time and labor are required in this method for conducting analysis over a wide region of DNA and determining mutation tendencies as a whole.

On the other hand, the method of the present invention is free from such determination of mutation sites based on the appearance frequencies of mismatched bases among read sequences. The method of the present invention basically involves obtaining mutation information based on the comparison of each of one or more read sequences corresponding to a specific site of a reference sequence, with the reference sequence, and optionally classifying the obtained information, and creating a database. The tendencies of mutations in the DNA to be analyzed are analyzed on the basis of the database. For example, statistical analysis using arbitrary components contained in the database as a sample population (e.g., mutation frequency analysis or mutation pattern analysis) can be conducted.

The method of the present invention is capable of detecting a low-frequency mutation without overlook because the respective mutation information is obtained as to each read sequence. Furthermore, the method of the present invention is capable of detecting and analyzing mutations in various regions on DNA, which correspond to any of the read sequences used in the mutation detection. Thus, the method of the present invention can detect mutations in a higher-throughput and more highly sensitive manner than the conventional methods and therefore achieves more efficient and more accurate mutation analysis.

### (2-1. Detection of Mutation in Each Fragment Using Next-Generation Sequencer)

In a preferred embodiment of the method of the present invention, procedures of sequencing DNA fragments using a next-generation sequencer which conducts PCR, and analyzing mutations in the DNA to be analyzed by comparison with the reference sequence will be described below in detail.

Adaptor sequences for sequencing are added to both ends of each fragment to be sequenced derived from the DNA to be analyzed. The adaptor-added fragment is amplified by PCR to an amount detectable by sequencing with a next-generation sequencer. The amplified fragment is sequenced, and the sequenced sequence is output as a read sequence. In a preferred embodiment of the present invention, two read sequences (read 1 and read 2) are obtained per amplified fragment. In this respect, read 1 corresponds to the sequence of the original fragment read by the sequencing, while read 2 corresponds to a complementary strand thereof. In the case of preparing a fragment having a size less than twice the read length of the sequencer, read 1 and read 2 of each amplified fragment contain at least a portion of the fragment as a common region and each further contain an upstream region or a downstream region thereof. One conjugated read sequence is constructed for each amplified fragment by merging the sequenced read 1 and read 2 at the common region. The construction of the conjugated read sequence from two read sequences can be carried out using software such as PEAR (Bioinformatics, 2014, 30 (5): 614-620), FLASH (Bioinformatics, 2011, 27 (21): 2957-2963), or PANDAseq (BMC Bioinformatics, 2012, 13: 31).

Subsequently, each read sequence is mapped onto the reference sequence for comparison. In a preferred embodiment, the comparison can be conducted only for the “consensus bases” mentioned above on the read sequence, for improvement in comparison accuracy. In another preferred embodiment, a conjugated read sequence is used as the read sequence in the comparison, for improvement in comparison accuracy. More preferably, a region to be compared with the reference sequence in the conjugated read sequence is restricted to the overlapping region of read 1 and read 2, and the bases on the conjugated read sequence for use in the comparison are restricted to bases complementary between read 1 and read 2 (i.e., the “consensus bases”). These procedures can reduce the adverse effects of a sequencing error on the sequence comparison. The restriction to the “consensus bases” may be carried out before mapping to the reference sequence or may be carried out after mapping to the reference sequence.

A site of a mismatch base (mutation site) in each read sequence with respect to the reference sequence can be detected by the mapping to and comparison with the reference sequence. Mutation information including the type of the mutation site (a substitution site, a deletion site, or an insertion site), the type of the base at the site, the type of a base before the mutation, the types of both adjacent bases of the site, etc. can be further obtained. These procedures are conducted on the read sequences derived from each original fragment obtained by the sequencing to collect mutation information. A database of the mutation information from each read sequence can be created. These procedures may be sequentially conducted on the read sequences one by one or may be conducted in parallel on a plurality of read sequences.

In the aforementioned procedures of mapping the read sequence to the reference sequence, restricting a region for comparison, marking a site of a mismatch base with respect to the reference sequence, and obtaining mutation information on the site, for example, the mapping can be carried out using Bowtie 2 software (Nature Methods, 2012, 9 (4): 357-359) or BWA software (Bioinformatics, 2009, 25 (14): 1754-1760); the restriction of a region for comparison and the marking of a mismatch base with respect to the reference sequence can be carried out using Samtools software (Bioinformatics, 2009, 25 (16): 2078-2079); and the obtainment of mutation information on the site can be carried out using, for example, a program to detect bases different from those of the reference sequence, which is created using a programming language such as Python. However, the software or the programming language for conducting the procedures for the method of the present invention is not limited thereto.

### (2-2. Mutation Analysis in Cell Population)

The tendencies of mutations in a cell population can be investigated on the basis of the obtained mutation information database. Preferably, examples of the mutations to be analyzed according to the present invention include base pair substitution mutations which change a base pair in DNA to a different base pair, and short insertion or deletion mutations which cause the insertion or deletion of a short nucleotide sequence in the sequence of DNA. Examples of the base pair substitution mutation include one-base pair substitution mutation, and multi-base pair substitution mutation such as two-base pair or three-base pair or more substitution. Among them, one-base pair substitution mutations are preferably analyzed in the present invention. According to the present invention, the mutation patterns and mutation frequencies of these mutations allow to be determined. Hereinafter, procedures for the analysis will be described in detail.

### (2-2-1. Analysis of Base Pair Substitution Mutations)

In one embodiment, a one-base pair substitution mutations are analyzed. In the present embodiment, as mentioned above, each of one or more read sequences obtained from each original fragment is compared with the reference sequence to detect a site of a base in each read sequence mismatched with respect to the reference sequence. The detected sites are obtained as mutation sites each having a base pair substitution mutation with respect to the reference sequence. Subsequently, each of the mutations is classified according to base mutation patterns on the basis of the type of the base at the detected mutation site and a base before the mutation. Subsequently, respective appearance frequencies of the base mutation patterns thus obtained are determined. These procedures can be conducted with, for example, the aforementioned program created using a programming language such as Python.

In a more specific example, the bases contained in the read sequences are divided into the following bases (i) to (iv):

(i) a base located at a position where the base on the reference sequence is A,

(ii) a base located at a position where the base on the reference sequence is T,

(iii) a base located at a position where the base on the reference sequence is G, and

(iv) a base located at a position where the base on the reference sequence is C.

The bases (i) and (ii) are bases present at sites where the base pair on the reference sequence is AT. The bases (iii) and (iv) are bases present at sites where the base pair on the reference sequence is GC. From among these bases, a mismatch base (i.e., base having a base pair substitution mutation) with respect to the reference sequence is detected. Subsequently, as to each of the mutated bases thus detected, a base pair before the mutation at the mutation site is determined from the base information on the reference sequence on the basis of the classification of the bases (i) to (iv), and a base pair after the mutation is also determined from the base information on each read sequence. From these data, each mutation can be classified into 6 base pair mutation patterns in total: 3 patterns when the base pair before the mutation is AT [AT→TA, AT→CG, and AT→GC]; and 3 patterns when the base pair before the mutation is GC [GC→TA, GC→CG, and GC→AT]. The appearance frequency of each of the mutation patterns can be determined on the basis of the total number of mutations belonging to the each of the mutation patterns and the total number of bases analyzed. For example, the appearance frequencies of 3 types of mutation patterns can be calculated for each of the AT and GC base pairs on the basis of the total number of bases analyzed on the each of the base pairs.

The mutation pattern of each mutation thus obtained can be further classified according to an original base. The original base at a mutation site where the base pair before the mutation is AT is A or T, and the original base at a mutation site where the base pair before the mutation is GC is G or C. Thus, each of the 6 base pair mutation patterns can be further classified into 2 groups according to an original base. Such classification is useful for removing a reading error of the sequencing due to base modification in the process of DNA extraction or isolation from cells. Particularly, it is known that the G base is susceptible to chemical modification by oxidation in the process of DNA preparation that causes error to misread G as T. Normally, since a mutation in base pair alters both bases constituting the base pair, mutation frequencies of the 2 groups of the base pair mutation pattern classified according to an original base are supposed to be equivalent. If the mutation frequency is biased to either of the original bases, this suggests a sequencing error attributed to base modification.

In an alternative embodiment of the present invention, multi-base pair substitution mutations are analyzed. Examples of the multi-base pair substitution mutation include two-base pair substitution mutation and three-base pair substitution mutation. In the case of analyzing multi-base pair substitution mutations, for example, each mutation pattern is classified according to a nucleotide sequence before the mutation (e.g., into 4×4=16 patterns for the two-base pair substitution). Subsequently, the appearance frequency of each mutation pattern can be determined on the basis of the total number of mutations belonging to the each mutation pattern and the total number of mutations analyzed.

### (2-2-2. Sequence Context Analysis)

In recent years, an approach of mathematically extracting a factor of mutation caused by a mutagen (mutation signature) from mutation information accumulated on the genome of cancer cells has been proposed. Various mutation signatures have been identified from mutation information accumulated in the genome in various human cancers (Cell Rep, 2013, 3: 246-0.259). In the theory of the mutation signatures, the mutation pattern of base pair substitution mutation is classified on the basis of a sequence context in upstream and downstream of the position of the base pair. The base pair substitution mutation can be analyzed in more detail by sequence context analysis.

Thus, in an alternative embodiment, procedures for the sequence context analysis of a one-base pair substitution mutation will be given below. In the present embodiment, as mentioned above, each read sequence is first compared with the reference sequence to detect a one-base pair substitution mutation in the read sequence. Subsequently, as to each detected mutation, a sequence (so-called context) including a base before the mutation and adjacent upstream and downstream bases of the base before the mutation is determined on the basis of the reference sequence. Subsequently, each mutation is typed according to base pair mutation patterns and the context. Specifically, the detected mutations are divided into 6 base pair mutation patterns [AT→TA, AT→CG, AT→GC, GC→TA, GC→CG, and GC→AT] by the same procedures as in the paragraph (2-2-1.) mentioned above. Meanwhile, each detected mutation is classified according to the context. For example, a context of 3 bases long including adjacent bases one upstream and one downstream of the mutation site is classified into 4×4=16 groups [e.g., ACA, ACC, ACG, ACT, CCA, CCC, CCG, CCT, GCA, GCC, GCG, GCT, TCA, TCC, TCG, and TCT for mutations from C]. As a result, each mutation is classified into 96 types (4×6×4) in total according to the base pair mutation patterns and the context. The sequence of the context for use in this analysis can consist of a base before the mutation, one or more adjacent upstream bases of the base before the mutation, and one or more adjacent downstream bases of the base before the mutation. Also, the length of the context can be 3 bases or more, though not limited thereto. If necessary, a longer context may be analyzed. For example, each mutation is classified into 256 groups (4×4×4×4) according to a context of 5 bases long including adjacent bases two upstream and two downstream of the mutation site. In this case, each mutation is finally classified into 1536 (4×4×6×4×4) types in total by the classification and 6 base pair patterns. Furthermore, each mutation is classified into 42n groups according to a context of 2n+1 bases long including adjacent bases n upstream and n downstream of the mutation site. In this case, each mutation is finally classified into 42n×6 types in total by the classification and 6 base pair patterns. Subsequently, respective mutation frequencies of these mutation types can be determined on the basis of the total number of mutations belonging to each of the mutation types and the total number of bases analyzed.

### (2-2-3. Analysis of Short Insertion or Deletion Mutations)

In a further alternative embodiment, short insertion or deletion mutations are analyzed. In the present embodiment, as mentioned above, each read sequence is compared with the reference sequence to detect a site where a base is inserted or deleted in the read sequence with respect to the reference sequence. The detected site is obtained as a mutation site having an insertion or deletion mutation with respect to the reference sequence. Further, as to each obtained mutation, the type of the mutation (an insertion mutation or a deletion mutation), the base length of the insertion or deletion site, and/or the type of the inserted or deleted base are determined. In the present embodiment, the insertion or deletion site to be detected is preferably a site having an inserted or deleted base length of 10 bp or less, more preferably 1 to 5 bp, though not limited thereto. The procedures of detecting an insertion or deletion site having a specific base length can be conducted using the aforementioned program created using a programming language such as Python. In addition, the type of the inserted or deleted base can be identified by the comparison of each read sequence with the reference sequence. The base length of an insertion or deletion site in each read sequence, and/or the type of the base at the insertion or deletion site can be determined by these procedures. Subsequently, a frequency of the insertion or deletion is determined for each of the base lengths and/or each of the types of the bases thus determined. For example, the insertion or deletion mutations obtained for respective read sequences can be classified by base length to determine their respective frequencies. For example, the inserted or deleted bases can be classified by type (A, T, G, and C) to determine their respective frequencies. Furthermore, mutations can be classified in more detail by combining the classification by base length and the classification by base type to determine their respective frequencies.

### (2-3. Analysis of Increased Mutation Frequency)

The mutations detected by the comparison of the read sequences with the reference sequence according to the procedures mentioned above may contain a base reading error of the sequencing. For more highly accurate mutation analysis, it is preferred to remove this error component. The removal of the error component can be conducted by subtracting mutation frequency of a control cell population from mutation frequency of the subject cell population. When the subject cell population is a cell population exposed to a specific condition, the influence of the specific condition on a mutation frequency may be analyzed by determining the difference in the mutation frequency between the subject cell population and the control. For example, when the subject cell population is a cell population exposed to a specific condition such as a cell population exposed to a mutagen or a cell population given a drug, the same cell population unexposed to the condition is used as the control cell population. This control cell population is analyzed by the same procedures as above for base pair substitution mutation, sequence context, or insertion or deletion mutation to determine mutation frequencies. The mutation frequency of the control cell population thus obtained is subtracted from the mutation frequency of the subject cell population. As a result, the sequencing error component is removed from the mutation frequency of the subject cell population, while the presence or absence of increase in the mutation frequency due to the specific condition, or an increased mutation frequency with respect to the control under the specific condition can be investigated. It is more preferred to analyze mutation frequencies on the basis of the aforementioned classification according to an original base, because a sequencing error attributed to base modification can be detected.

### 3. Application

The aforementioned method for analyzing mutations according to the present invention is capable of quantitatively and qualitatively analyzing mutations in a cell population. The analysis method of the present invention can be applied to various analyses or evaluations associated with mutations. Typical examples of the application include methods for evaluating the genotoxicity of a substance, methods for evaluating tumorigenic mutations (e.g., methods for evaluating mutations in cancer cells, and evaluating mutations in cfDNA), and the quality control of cultured cells (e.g., the evaluation of genetic information such as the evaluation of the presence or absence of mutations, or mutation types).

Thus, in a preferred embodiment, the present invention provides a method for evaluating the genotoxicity of a test substance. Specific procedures for the method will be described below.

### (3-1. Method for Evaluating Genotoxicity of Substance)

### (3-1-1. Evaluation Based on Analysis of Base Pair Substitution Mutation)

In one embodiment, the method for evaluating the genotoxicity of a test substance according to the present invention is based on the analysis of a base pair substitution mutation mentioned above. This method comprises:

(1) obtaining DNAs from a test group, the test group is a cell population exposed to the test substance;
(2) sequencing fragments of the DNAs to obtain one or more read sequences per fragment;
(3) comparing each of the one or more read sequences with a reference sequence to detect sites of mismatch bases between the each read sequence and the reference sequence, wherein the reference sequence is a known sequence in the DNAs;
(4) obtaining the sites detected in the step (3) as sites of mutation each having a base pair substitution mutation;
(5) classifying each of the obtained mutations according to base pair mutation patterns; and
(6) determining respective mutation frequencies of the mutation patterns obtained in the step (5).

The steps (1) to (6) are as mentioned above in the paragraph (2.), particularly, the paragraph (2-2-1.). More specifically, in the case of analyzing one-base pair substitution mutations, in the step (5), each mutation is classified into 3 mutation patterns [AT→TA, AT→CG, and AT→GC] as to sites where the base pair on the reference sequence is AT, and classified into 3 mutation patterns [GC→TA, GC→CG, and GC→AT] as to sites where the base pair on the reference sequence is GC. Preferably, these classifications are combined and mutations are classified into 6 base pair mutation patterns in total. Each of these 6 base pair mutation patterns is further divided into 2 groups on the basis of the types of the original bases (A or T for AT, or G or C for GC). Thus, mutations may be classified into 12 mutation patterns in total. Subsequently, in the step (6), respective mutation frequencies of the mutation patterns determined in the step (5) are determined. As a result, the mutation frequency of each base pair mutation pattern can be determined.

In a preferred embodiment, the method described above further comprises:

(7) conducting the same procedures as in the steps (1) to (6) on a control group, the control group is a cell population unexposed to the test substance, thereby determining respective mutation frequencies of the base pair mutation patterns in the control group; and
(8) subtracting the respective mutation frequencies of the mutation patterns in the control group obtained in the step (7) from the respective mutation frequencies of the mutation patterns in the test group obtained in the step (6).

As a result, increased mutation frequencies in the test group without being influenced by sequencing error can be determined.

### (3-1-2. Evaluation Based on Sequence Context Analysis)

In a further embodiment, the method for evaluating the genotoxicity of a test substance according to the present invention is based on the sequence context analysis mentioned above. This method comprises:

(1′) obtaining DNAs from a test group, the test group is a cell population exposed to the test substance;
(2′) sequencing fragments of the DNAs to obtain one or more read sequences per fragment;
(3′) comparing each of the one or more read sequences with a reference sequence to detect sites of mismatch bases between the each read sequence and the reference sequence, wherein the reference sequence is a known sequence in the DNAs;
(4′) obtaining the sites detected in the step (3′) as sites of mutation each having a base pair substitution mutation;
(5′) as to each of the mutations thus obtained, determining a context sequence comprising a base before mutation and adjacent upstream and downstream bases of the base before mutation, the context sequence is determined on the basis of the reference sequence;
(6′) typing each of the mutations obtained in the step (4′) according to the context sequence determined in the step (5′) and the type of the base after mutation; and
(7′) determining respective mutation frequencies of the mutation types obtained in the step (6′).

The steps (1′) to (7′) are as mentioned above in the paragraph (2.), particularly, the paragraph (2-2-2.). More specifically, in the step (6′), each mutation is classified into 96 types in total on the basis of the 6 base pair mutation patterns mentioned above [AT→TA, AT→CG, AT→GC, GC→TA, GC→CG, and GC→AT] and 16 groups according to the types of both adjacent bases of the mutation site [e.g., ACA, ACC, ACG, ACT, CCA, CCC, CCG, CCT, GCA, GCC, GCG, GCT, TCA, TCC, TCG, and TCT for mutations from C]. Subsequently, in the step (7′), respective mutation frequencies of the mutation types determined in the step (6′) are determined. As a result, mutation types and mutation frequencies can be determined.

In a preferred embodiment, the method described above further comprises:

(8′) conducting the same procedures as in the steps (1′) to (7′) on a control group, the control group is a cell population unexposed to the test substance, thereby determining respective mutation frequencies of the mutation types in the control group; and
(9′) subtracting the respective mutation frequencies of the mutation types in the control group obtained in the step (8′) from the respective mutation frequencies of the mutation types in the test group obtained in the step (7′).

As a result, increased mutation frequencies in the test group without being influenced by sequencing error can be determined.

### (3-1-3. Evaluation Based on Analysis of Short Insertion or Deletion Mutation)

In a further embodiment, the method for evaluating the genotoxicity of a test substance according to the present invention is based on the analysis of a short insertion or deletion mutation mentioned above. This method comprises:

(1″) obtaining DNAs from a test group, the test group is a cell population exposed to the test substance;
(2″) sequencing fragments of the DNAs to obtain one or more read sequences per fragment;
(3″) comparing each of the one or more read sequences with a reference sequence to detect sites of base insertion or deletion in the each read sequence with respect to the reference sequence, wherein the reference sequence is a known sequence in the DNAs;
(4″) obtaining the sites detected in the step (3″) as sites of mutation each having an insertion or deletion mutation;
(5″) as to each of the mutations thus obtained, determining the base length of the insertion or deletion and/or the type of the inserted base; and
(6″) determining respective mutation frequencies for the sites of the insertion or deletion mutations with different base lengths of the insertion or deletion and/or different types of the inserted bases determined in the step (5″).

The steps (1″) to (6″) are as mentioned above in the paragraph (2.), particularly, the paragraph (2-2-3.). More specifically, in the step (3″), a site of insertion or deletion in each read sequence wherein base length of the insertion or deletion is preferably 10 bp or less, more preferably 1 to 5 bp, is detected by comparison with the reference sequence. When an insertion or deletion has been detected, the type of the inserted or deleted base can be further identified by the comparison of each read sequence with the reference sequence. As a result, the base length of the insertion or deletion site in the each read sequence, and/or the type of the inserted or deleted base can be determined (step (5″)). Subsequently, in the step (6″), respective mutation frequencies are determined for the sites of the insertion or deletion mutations with different base lengths of the insertion or deletion and/or different types of the inserted or deleted bases determined in the step (5″). As a result, mutation patterns and mutation frequencies can be determined.

In a preferred embodiment, the method described above further comprises:

(7″) conducting the same procedures as in the steps (1″) to (6″) on a control group, the control group is a cell population unexposed to the test substance, thereby determining respective mutation frequencies for each of the sites of the insertion or deletion mutations with different base lengths of the insertion or deletion and/or different types of the inserted or deleted bases in the control group; and
(8″) subtracting the respective mutation frequencies in the control group obtained in the step (7″) from the respective mutation frequencies for each of the sites of the insertion or deletion mutations with different base lengths of the insertion or deletion and/or different types of the inserted or deleted bases in the test group obtained in the step (6″).

As a result, increased mutation frequencies in the test group without being influenced by sequencing error can be determined.

The method for evaluating the genotoxicity of a test substance according to the present invention is capable of analyzing the tendencies of mutations at a cell population level as to a population of cells which might have different mutations by exposure to a test substance. Thus, according to the present invention, information which reflects better the actual influence of the test substance in vivo can be obtained as compared with the conventional single cell-based analysis methods (e.g., Non Patent Literatures 4 and 5). Furthermore, according to the present invention, the influence of the test substance on a cell population can be quantitatively and qualitatively analyzed at a level of individual bases on DNA. Hence, more detailed information on the genotoxicity of the test substance can be obtained.

### (3-2. Method for Evaluating Tumorigenic Mutation)

### (3-2-1. Method for Evaluating Mutation in Cancer Cell)

In an alternative preferred embodiment, the present invention provides a method for evaluating mutations in cancer cells. Specific procedures for the method are basically the same as in the aforementioned method for evaluating the genotoxicity of a test substance except that a population of cancer cells is used as the test group. Alternatively, a population of cells suspected of having cancer or cells to be evaluated for the risk of cancerization may be used as the test group, instead of the cancer cell population. A population of non-cancer cells (e.g., normal cells) or a population of cells with a low risk of cancerization is used as the control group. This method is useful for identifying the tendencies of cancer type-specific mutations, evaluating the risk of cancerization, or confirming the stage of progression or malignancy of cancer. As for the evaluation of mutations in cancer cells, more detailed analysis is achieved by the sequence context analysis as described in the paragraph (2-2-2.). It has previously been reported that in the genome analysis of human cancers, mutations were classified into 96 groups (4×6×4) on the basis of 3-base contexts or 1536 groups (4×4×6×4×4) on the basis of 5-base contexts (Cell Rep, 2013, 3: 246-259).

The method for evaluating mutations in cancer cells according to the present invention is capable of identifying the tendencies of the cancer type-specific mutations in the whole cancer cell population, unlike the conventional methods for analyzing mutations at a specific site on the genome, i.e., methods for extracting only mutations which appear at a given or higher rate in cells in cancer tissues by the selection in the cancer tissues, such as methods which involve aligning and comparing a plurality of read sequences at the same location on the genome (e.g., Non Patent Literatures 4 and 5). Mutations in cancer cells reportedly vary in quantity or quality depending on the type of cancer (Nature, 2013, 500 (7463): 415-421). The method of the present invention is capable of quantitatively and qualitatively analyzing the tendencies of mutations in a cancer cell population and is therefore supposed to be useful in the diagnosis of the progression stage or type of cancer.

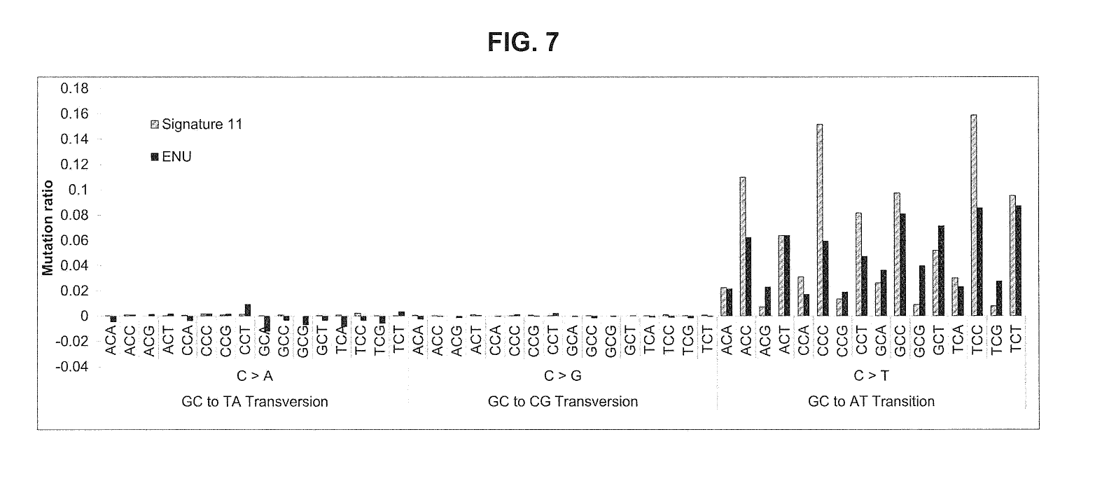

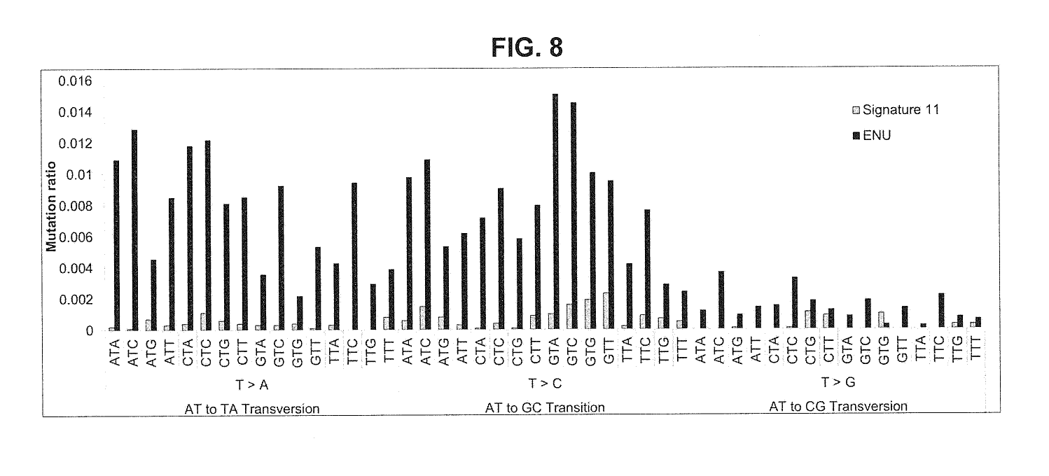

The conventional analysis of mutation signatures involves: sequencing genomic DNA obtained from each person within a population considered to have tumor induced by a specific cause; extracting mutations which appear at a given or higher rate in cancer tissues according to a conventional method (e.g., Non Patent Literature 4 or 5); then conducting sequence context analysis on the extracted mutations; subsequently compiling mutation information obtained from each person; and identifying mutation signatures within the population. However, in the case of analyzing a mutation signature in the diagnosis of an individual, it might be difficult to determine the similarity of the obtained sequence contexts to known mutation signatures, due to a small number of mutations identified. On the other hand, according to the present invention, sequence context data having quality that permits confirmation of similarity to mutation signatures can be obtained by single next-generation sequencing analysis, as shown in FIG. 7 and FIG. 8. This is because, unlike the conventional methods, the number of mutations sufficient for sequence context analysis can be secured by adding to analytes mutations which fall short of the given or higher rate. Thus, the method of the present invention is considered exploitable in the high-throughput sequence context analysis of mutations in an individual.

(3-2-2. Method for Evaluating Mutation in cfDNA)

In an alternative preferred embodiment, the present invention provides the analysis of cell-free DNA (cfDNA) in blood. The cfDNA has received attention as to a low invasive method for diagnosing tumorigenesis in humans. The cfDNA is obtained from biopsy from liquid such as plasma, serum, or urine. Specific procedures for the application of the method to the cfDNA analysis are basically the same as in the aforementioned method for evaluating the genotoxicity of a test substance except that cfDNA in liquid biopsies harvested from human cancer patients or the like is used as the DNA of the test group, instead of the DNA obtained from the cancer cell population. For example, cfDNA derived from healthy humans or cfDNA harvested from the same humans in advance before development of cancer is used as the control group. This method is useful for identifying the tendencies of cancer patient-specific mutations in cfDNA or confirming the progression stage or malignancy of cancer.

The method for evaluating mutations in cfDNA according to the present invention is capable of identifying the tendencies of the cancer type-specific mutations in the whole cancer cell population, unlike the conventional analysis methods, i.e., methods for extracting only mutations occupying a given proportion in cfDNA and predicted to appear at a given rate in cells in cancer tissues, such as methods which involve adding a unique tag sequence to each molecule of cfDNA, obtaining consensus sequences of a plurality of read sequences obtained from the same molecule, and then aligning and comparing the plurality of read sequences at the same location on the genome (see Non Patent Literatures 6 and 7). As for the evaluation of mutations in cfDNA, more detailed analysis is achieved by the sequence context analysis as described in the paragraph (2-2-2.). The method of the present invention is low invasive and is useful for, for example, identifying the tendencies of cancer cell-specific mutations, confirming the progression stage or malignancy of cancer, or detecting microscopic tumor at an early stage of progression. Thus, the method of the present invention may be applied to routine examination of cancer, medical checkup, etc.

### (3-3. Method for Evaluating Genetic Information in Cultured Cell)

In an alternative preferred embodiment, the present invention provides a method for evaluating genetic information in cultured cells. Specific procedures for the method are basically the same as in the aforementioned method for evaluating the genotoxicity of a test substance except that a population of cultured cells to be examined for the presence or absence of mutations is used as the test group. Examples of the test group include cells subcultured for a certain period, wherein the tendencies of mutations therein are to be confirmed. A population of cells which are cultured cells of the same type as in the test group and have known genetic information (e.g., cells wherein the presence or absence of mutations and mutation types thereof have already been confirmed) is used as the control group. Examples of the control group include cells before subculture. This method allows to evaluate the presence or absence of mutations in cultured cells or mutation types thereof. The method is useful for the quality control of cultured cells.

The method for evaluating genetic information in cultured cells according to the present invention is capable of identifying the tendencies of mutations specific for the cultured cells in the cultured cell population, rather than mutations in individual cells. This method allows to evaluate whether or not cultured cells such as iPS cells retain genetic quality (whether or not the cultured cells have mutations). For example, genetic quality control in the preparation of human-derived iPS cells is very important for the clinical application thereof. It has been reported that various mutations occur in the genome of iPS cells in the process of their establishment. These mutations might lead to carcinogenesis, etc. after transplantation to patients. Thus, genetic quality control thereof is essential (Nature, 2011, 471 (7336): 63-67). Use of the method of the present invention permits convenient grasping of the tendencies of mutations in a population of iPS cells. Furthermore, the method of the present invention is a highly comprehensive method as compared with a generally-using conventional method for evaluating the quality of iPS cells by use of PCR, and can be very inexpensive as compared with another conventional method for tumorigenesis using SCID mice (PLoS One, 2012, 7 (5): e37342). Thus, the method of the present invention may also be useful as a convenient and inexpensive screening approach for the genetic quality control of iPS cells.

### (3.4. Various Conditions)

In any of the embodiments described above, a known sequence in the DNA of a cell population of a test group can be used as the reference sequence. It is preferred to use a sequence registered in a public database or the like as the reference sequence. A sequence in the genomic DNA of the cell population, sequenced in advance using a sequencer or the like prior to the method of the present invention may be used as the reference sequence.

The test substance used in the method for evaluating the genotoxicity of a test substance according to the present invention is not particularly limited as long as the substance is to be evaluated for its genotoxicity. Examples thereof include substances suspected of having genotoxicity, substances for which the presence or absence of genotoxicity is to be confirmed, and substances to be examined for the types of inducible mutations. The test substance may be a naturally occurring substance or may be a substance artificially synthesized by a chemical or biological method or the like. The test substance may be a compound or may be a composition or a mixture. Alternatively, the test substance may be ultraviolet ray, radiation, or the like.

The approach of exposing the cell population to the test substance is not particularly limited and can be appropriately selected according to the type of the test substance. Examples thereof include a method of adding the test substance to a medium containing the cell population, and a method of leaving the cell population in an atmosphere containing the test substance.

Examples of the cell population used in the method of the present invention include specimens harvested from animals or plants, and populations of animal-, plant- or microbe-derived cultured cells and preferably include populations of animal, plant or microbe strain-derived cultured cells. Examples of the animal preferably include mammals such as humans, silkworms, and nematodes. Examples of the microbes preferably include E. coli, Salmonella, and yeasts.

Among the cell populations listed above, preferably, a population of microbe strain-derived cultured cells is used in the method for evaluating the genotoxicity of a test substance. More preferably, at least one cell population selected from the group consisting of a population of E. coli cells and a population of Salmonella cells is used. Preferred examples of the Salmonella include a S. typhimurium LT-2 strain, and S. typhimurium TA100 strain, TA98 strain, TA1535 strain, TA1538 strain, and TA1537 strain which are used in the Ames test. Preferred examples of the E. coli include a WP2 strain and a WP2 uvrA strain which are also used in the Ames test.

Examples of the type of the cancer cells which can be used in the present invention include, but are not particularly limited to, cells of lung cancer, breast cancer, prostate cancer, tongue cancer, laryngeal or pharyngeal cancer, digestive organ cancer (e.g., esophageal cancer, stomach cancer, duodenum cancer, large intestine cancer, colon or rectum cancer, etc.), liver cancer, pancreatic cancer, cervical cancer, uterine corpus cancer, renal cell cancer, renal pelvic cancer, bladder cancer, brain tumor, bone tumor, leukemia, lymphoma, myeloma, skin cancer, and malignant melanoma. These cancer cells may be derived from specimens harvested from animals or may be a cultured cancer cell line.

The present specification further discloses the following substances, production methods, use, methods, etc. as exemplary embodiments of the present invention. However, the present invention is not limited by these embodiments.

[1] A method for evaluating the genotoxicity of a test substance, comprising:
(1) obtaining DNAs from a test group, the test group is a cell population exposed to the test substance;
(2) sequencing fragments of the DNAs to obtain one or more read sequences per fragment;
(3) comparing each of the one or more read sequences with a reference sequence to detect sites of mismatch bases between the each read sequence and the reference sequence, wherein the reference sequence is a known sequence in the DNAs;
(4) obtaining the sites detected in the step (3) as sites of mutation each having a base pair substitution mutation;
(5) classifying each of the obtained mutations according to base pair mutation patterns; and
(6) determining respective mutation frequencies of the mutation patterns obtained in the step (5).
[2] The method according to [1], preferably, further comprising

extracting, from the bases of the read sequences obtained in the step (2), bases with high reading reliability of the sequencing, wherein

the step (3) comprises comparing the extracted bases on the read sequences with the bases of the reference sequence.

[3] The method according to [1], preferably, further comprising extracting bases with high reading reliability of the sequencing among the bases of the sites detected by the comparison of the read sequences with the reference sequence in the step (3).
[4] The method according to any one of [1] to [3], wherein preferably, the steps (3) to (5) comprise:

dividing bases contained in the read sequences into the following bases (i) to (iv):
(i) a base located at a position where the base on the reference sequence is A,
(ii) a base located at a position where the base on the reference sequence is T,
(iii) a base located at a position where the base on the reference sequence is G, and
(iv) a base located at a position where the base on the reference sequence is C;

detecting mismatch bases with respect to the reference sequence from among the bases contained in the read sequences, and obtaining sites of the detected bases as sites of mutation each having a base pair substitution mutation;

obtaining base pairs before mutation and after mutation at the respective sites of mutation of the detected mismatch bases; and

classifying the base pair substitution mutations at the sites of mutation, according to the types of the base pairs before mutation and the base pairs after mutation, into 6 of base pair mutation patterns: AT→TA, AT→CG, AT→GC, GC→TA, GC→CG, and GC→AT.

[5] The method according to [4], preferably, further comprising classifying each of the 6 base pair mutation patterns into 2 groups according to an original base.
[6] The method according to any one of [1] to [5], preferably, further comprising:
(7) conducting the same procedures as in the steps (1) to (6) on a control group, the control group is a cell population unexposed to the test substance, thereby determining respective mutation frequencies of the base pair mutation patterns in the control group; and
(8) subtracting the respective mutation frequencies of the mutation patterns in the control group obtained in the step (7) from the respective mutation frequencies of the mutation patterns in the test group obtained in the step (6).
[7] A method for evaluating the genotoxicity of a test substance, comprising:
(1′) obtaining DNAs from a test group, the test group is a cell population exposed to the test substance;
(2′) sequencing fragments of the DNAs to obtain one or more read sequences per fragment;
(3′) comparing each of the one or more read sequences with a reference sequence to detect sites of mismatch bases between the each read sequence and the reference sequence, wherein the reference sequence is a known sequence in the DNAs;
(4′) obtaining the sites detected in the step (3′) as sites of mutation each having a base pair substitution mutation;
(5′) as to each of the mutations thus obtained, determining a context sequence comprising a base before mutation and adjacent upstream and downstream bases of the base before mutation, the context sequence is determined on the basis of the reference sequence;
(6′) typing each of the mutations obtained in the step (4′) according to the context sequence determined in the step (5′) and the type of the base after mutation; and
(7′) determining respective mutation frequencies of the mutation types obtained in the step (6′).
[8] The method according to [7], preferably, further comprising

extracting, from the bases of the read sequences obtained in the step (2′), bases with high reading reliability of the sequencing, wherein

the step (3′) comprises comparing the extracted bases on the read sequences with the bases of the reference sequence.

[9] The method according to [7], preferably, further comprising extracting bases with high reading reliability of the sequencing among the bases of the sites detected by the comparison of the read sequences with the reference sequence in the step (3′).
[10] The method according to any one of [7] to [9], wherein preferably, the steps (3′) to (6′) comprise:

dividing bases contained in the read sequences into the following bases (i) to (iv):)
(i) a base located at a position where the base on the reference sequence is A,
(ii) a base located at a position where the base on the reference sequence is T,
(iii) a base located at a position where the base on the reference sequence is G, and
(iv) a base located at a position where the base on the reference sequence is C;

detecting mismatch bases with respect to the reference sequence from among the bases contained in the read sequences, and obtaining sites of the detected bases as sites of mutation each having a base pair substitution mutation;

obtaining base pairs before mutation and after mutation at the respective sites of mutation of the detected mismatch bases;

classifying the base pair substitution mutations at the sites of mutation, according to the types of the base pairs before mutation and the base pairs after mutation, into 6 of base pair mutation patterns: AT→TA, AT→CG, AT→GC, GC→TA, GC→CG, and GC→AT;

determining context sequences each consisting of a base before mutation located at each of the sites of mutation, one or more adjacent upstream bases of the base before mutation, and one or more adjacent downstream bases of the base before mutation; and

typing each of the base pair substitution mutations according to the 6 of base pair mutation patterns and the context sequences.

[11] The method according to [10], wherein preferably, the context sequence is a sequence of 3 bases long including the base before mutation at the mutation site and adjacent bases one upstream and one downstream thereof, and the base pair substitution mutations are typed into 96 groups according to the 6 of base pair mutation patterns and the context sequences of 3 bases long.
[12] The method according to any one of [7] to [11], preferably, further comprising:
(8′) conducting the same procedures as in the steps (1′) to (7′) on a control group, the control group is a cell population unexposed to the test substance, thereby determining respective mutation frequencies of the mutation types in the control group; and
(9′) subtracting the respective mutation frequencies of the mutation types in the control group obtained in the step (8′) from the respective mutation frequencies of the mutation types in the test group obtained in the step (7′).
[13] A method for evaluating the genotoxicity of a test substance, comprising:
(1″) obtaining DNAs from a test group, the test group is a cell population exposed to the test substance;
(2″) sequencing fragments of the DNAs to obtain one or more read sequences per fragment;
(3″) comparing each of the one or more read sequences with a reference sequence to detect sites of base insertion or deletion in the each read sequence with respect to the reference sequence, wherein the reference sequence is a known sequence in the DNAs;
(4″) obtaining the sites detected in the step (3″) as sites of mutation each having an insertion or deletion mutation;
(5″) as to each of the mutations thus obtained, determining the base length of the insertion or deletion and/or the type of the inserted or deleted base; and
(6″) determining respective mutation frequencies for each of the sites of the insertion or deletion mutations with different base lengths of the insertion or deletion and/or different types of the inserted or deleted bases determined in the step (5″).
[14] The method according to [13], preferably, further comprising

extracting, from the bases of the read sequences obtained in the step (2″), bases with high reading reliability of the sequencing, wherein

the step (3″) comprises comparing the extracted bases on the read sequences with the bases of the reference sequence.

[15] The method according to [13], preferably, further comprising extracting bases with high reading reliability of the sequencing among the bases of the sites detected by the comparison of the read sequences with the reference sequence in the step (3″).
[16] The method according to any one of [13] to [15], wherein the base length of the sites of the insertion or deletion to be detected is preferably 10 bp or less, more preferably 1 to 5 bp.
[17] The method according to any one of [13] to [16], preferably, further comprising:
(7″) conducting the same procedures as in the steps (1″) to (6″) on a control group, the control group is a cell population unexposed to the test substance, thereby determining respective mutation frequencies for each of the sites of insertion or deletion mutations with different base lengths of the insertion or deletion and/or different types of the inserted or deleted bases in the control group; and
(8″) subtracting the respective mutation frequencies in the control group obtained in the step (7″) from the respective mutation frequencies for each of the sites of the insertion or deletion mutations with different base lengths of the insertion or deletion and/or different types of the inserted or deleted bases in the test group obtained in the step (6″).
[18] The method according to any one of [1] to [12], wherein preferably, the base pair substitution mutation is a one-base pair substitution mutation, a two-base pair substitution mutation, or a three-base pair substitution mutation.
[19] The method according to any one of [1] to [18], wherein preferably, the cell population is at least one cell population selected from the group consisting of a Salmonella cell population and an E. coli cell population.
[20] The method according to [19], wherein preferably, the Salmonella is a S. typhimurium LT-2 strain, TA100 strain, TA98 strain, TA1535 strain, TA1538 strain or TA1537 strain.
[21] A method for evaluating mutations in cancer cells, comprising:
(1) obtaining DNAs from a test group, the test group is a cancer cell population;
(2) sequencing fragments of the DNAs to obtain one or more read sequences per fragment;
(3) comparing each of the one or more read sequences with a reference sequence to detect sites of mismatch bases between the each read sequence and the reference sequence, wherein the reference sequence is a known sequence in the DNAs;
(4) obtaining the sites detected in the step (3) as sites of mutation each having a base pair substitution mutation;
(5) classifying each of the obtained mutations according to base pair mutation patterns; and
(6) determining respective mutation frequencies of the mutation patterns obtained in the step (5).
[22] A method for evaluating genetic information in cultured cells, comprising:
(1) obtaining DNAs from a test group, the test group is a cultured cell population;
(2) sequencing fragments of the DNAs to obtain one or more read sequences per fragment;
(3) comparing each of the one or more read sequences with a reference sequence to detect sites of mismatch bases between the each read sequence and the reference sequence, wherein the reference sequence is a known sequence in the DNAs;
(4) obtaining the sites detected in the step (3) as sites of mutation each having a base pair substitution mutation;
(5) classifying each of the obtained mutations according to base pair mutation patterns; and
(6) determining respective mutation frequencies of the mutation patterns obtained in the step (5).
[23] The method according to [21] or [22], preferably, further comprising

extracting, from the bases of the read sequences obtained in the step (2), bases with high reading reliability of the sequencing, wherein

the step (3) comprises comparing the extracted bases on the read sequences with the bases of the reference sequence.

[24] The method according to [21] or [22], preferably, further comprising extracting bases with high reading reliability of the sequencing among the bases of the sites detected by the comparison of the read sequences with the reference sequence in the step (3).
[25] The method according to any one of [21] to [24], wherein preferably, the steps (3) to (5) comprise:

dividing bases contained in the read sequences into the following bases (i) to (iv):
(i) a base located at a position where the base on the reference sequence is A,
(ii) a base located at a position where the base on the reference sequence is T,
(iii) a base located at a position where the base on the reference sequence is G, and
(iv) a base located at a position where the base on the reference sequence is C;

detecting mismatch bases with respect to the reference sequence from among the bases contained in the read sequences, and obtaining sites of the detected bases as sites of mutation each having a base pair substitution mutation;

obtaining base pairs before mutation and after mutation at the respective sites of mutation of the detected mismatch bases; and

classifying the base pair substitution mutations at the sites of mutation, according to the types of the base pairs before mutation and the base pairs after mutation, into 6 of base pair mutation patterns: AT→TA, AT→CG, AT→GC, GC→TA, GC→CG, and GC→AT.

[26] The method according to [25], preferably, further comprising classifying each of the 6 base pair mutation patterns into 2 groups according to an original base.
[27] The method according to any one of [21] to [26], preferably, further comprising:
(7) determining respective mutation frequencies of base pair mutation patterns in a control group by the same procedures as in the steps (1) to (6); and
(8) subtracting the respective mutation frequencies of the mutation patterns in the control group obtained in the step (7) from the respective mutation frequencies of the mutation patterns in the test group obtained in the step (6).
[28] The method according to [27], wherein preferably,

the method is a method for evaluating mutations in cancer cells, wherein the control group is a non-cancer cell population, or

the test group is a population of cells suspected of having cancer or cells to be evaluated for the risk of cancerization, the control group is a non-cancer cell population or a population of cells with a low risk of cancerization, and the risk of cancerization in the cells is evaluated by the method.

[29] The method according to [27], wherein preferably, the method is a method for evaluating genetic information in cultured cells, wherein the control group is a population of cells which are cultured cells of the same type as in the test group and have known genetic information.
[30] A method for evaluating mutations in cancer cells, comprising:
(1′) obtaining DNAs from a test group, the test group is a cancer cell population;
(2′) sequencing fragments of the DNAs to obtain one or more read sequences per fragment;
(3′) comparing each of the one or more read sequences with a reference sequence to detect sites of mismatch bases between the each read sequence and the reference sequence, wherein the reference sequence is a known sequence in the DNAs;
(4′) obtaining the sites detected in the step (3′) as sites of mutation each having a base pair substitution mutation;
(5′) as to each of the mutations thus obtained, determining a context sequence comprising a base before mutation and adjacent upstream and downstream bases of the base before mutation, the context sequence is determined on the basis of the reference sequence;
(6′) typing each of the mutations obtained in the step (4′) according to the context sequence determined in the step (5′) and the type of the base after mutation; and
(7′) determining respective mutation frequencies of the mutation types obtained in the step (6′).
[31] A method for evaluating genetic information in cultured cells, comprising:
(1′) obtaining DNAs from a test group, the test group is a cultured cell population;
(2′) sequencing fragments of the DNAs to obtain one or more read sequences per fragment;
(3′) comparing each of the one or more read sequences with a reference sequence to detect sites of mismatch bases between the each read sequence and the reference sequence, wherein the reference sequence is a known sequence in the DNAs;
(4′) obtaining the sites detected in the step (3′) as sites of mutation each having a base pair substitution mutation;
(5′) as to each of the mutations thus obtained, determining a context sequence comprising a base before mutation and adjacent upstream and downstream bases of the base before mutation, the context sequence is determined on the basis of the reference sequence;
(6′) typing each of the mutations obtained in the step (4′) according to the context sequence determined in the step (5′) and the type of the base after mutation; and
(7′) determining respective mutation frequencies of the mutation types obtained in the step (6′).
[32] The method according to [30] or [31], preferably, further comprising

extracting, from the bases of the read sequences obtained in the step (2′), bases with high reading reliability of the sequencing, wherein

the step (3′) comprises comparing the extracted bases on the read sequences with the bases of the reference sequence.

[33] The method according to [30] or [31], preferably, further comprising extracting bases with high reading reliability of the sequencing among the bases of the sites detected by the comparison of the read sequences with the reference sequence in the step (3′).
[34] The method according to any one of [30] to [33], wherein preferably, the steps (3′) to (6′) comprise:

dividing bases contained in the read sequences into the following bases (i) to (iv):
(i) a base located at a position where the base on the reference sequence is A,
(ii) a base located at a position where the base on the reference sequence is T,
(iii) a base located at a position where the base on the reference sequence is G, and
(iv) a base located at a position where the base on the reference sequence is C;

detecting mismatch bases with respect to the reference sequence from among the bases contained in the read sequences, and obtaining sites of the detected bases as sites of mutation each having a base pair substitution mutation;

obtaining base pairs before mutation and after mutation at the respective sites of mutation of the detected base mismatch;

classifying the base pair substitution mutations at the sites of mutation, according to the types of the base pairs before mutation and the base pairs after mutation, into 6 of base pair mutation patterns: AT→TA, AT→CG, AT→GC, GC→TA, GC→CG, and GC→AT;

determining context sequences each consisting of a base before mutation located at each of the sites of mutation, one or more adjacent upstream bases of the base before mutation, and one or more adjacent downstream bases of the base before mutation; and

typing each of the base pair substitution mutations according to the 6 of base pair mutation patterns and the context sequences.

[35] The method according to [34], wherein preferably, the context sequence is a sequence of 3 bases long including the base before mutation at the mutation site and adjacent bases one upstream and one downstream thereof, and the base pair substitution mutations are typed into 96 groups according to the 6 base pair mutation patterns and the context sequences of 3 bases long.
[36] The method according to any one of [30] to [35], preferably, further comprising:
(8′) determining respective mutation frequencies of mutation types in a control group by the same procedures as in the steps (1′) to (7′); and
(9′) subtracting the respective mutation frequencies of the mutation types in the control group obtained in the step (8′) from the respective mutation frequencies of the mutation types in the test group obtained in the step (7′).
[37] The method according to [36], wherein preferably,

the method is a method for evaluating mutations in cancer cells, wherein the control group is a non-cancer cell population, or

the test group is a population of cells suspected of having cancer or cells to be evaluated for the risk of cancerization, the control group is a non-cancer cell population or a population of cells with a low risk of cancerization, and the risk of cancerization in the cells is evaluated by the method.

[38] The method according to [36], wherein preferably, the method is a method for evaluating genetic information in cultured cells, wherein the control group is a population of cells which are cultured cells of the same type as in the test group and have known genetic information.
[39] A method for evaluating mutations in cancer cells, comprising:
(1″) obtaining DNAs from a test group, the test group is a cancer cell population;
(2″) sequencing fragments of the DNAs to obtain one or more read sequences per fragment;
(3″) comparing each of the one or more read sequences with a reference sequence to detect sites of base insertion or deletion in the each read sequence with respect to the reference sequence, wherein the reference sequence is a known sequence in the DNAs;
(4″) obtaining the sites detected in the step (3″) as sites of mutation each having an insertion or deletion mutation;
(5″) as to each of the mutations thus obtained, determining the base length of the insertion or deletion and/or the type of the inserted or deleted base; and
(6″) determining respective mutation frequencies for each of the sites of the insertion or deletion mutations with different base lengths of the insertion or deletion and/or different types of the inserted or deleted bases determined in the step (5″).
[40] A method for evaluating genetic information in cultured cells, comprising:
(1″) obtaining DNAs from a test group, the test group is a cultured cell population;
(2″) sequencing fragments of the DNAs to obtain one or more read sequences per fragment;
(3″) comparing each of the one or more read sequences with a reference sequence to detect sites of base insertion or deletion in the each read sequence with respect to the reference sequence, wherein the reference sequence is a known sequence in the DNAs;
(4″) obtaining the sites detected in the step (3″) as sites of mutation each having an insertion or deletion mutation;
(5″) as to each of the mutations thus obtained, determining the base length of the insertion or deletion and/or the type of the inserted or deleted base; and
(6″) determining respective mutation frequencies for each of the sites of the insertion or deletion mutations with different base lengths of the insertion or deletion and/or different types of the inserted or deleted bases determined in the step (5″).
[41] The method according to [39] or [40], preferably, further comprising

extracting, from the bases of the read sequences obtained in the step (2″), bases with high reading reliability of the sequencing, wherein

the step (3″) comprises comparing the extracted bases on the read sequences with the bases of the reference sequence.

[42] The method according to [39] or [40], preferably, further comprising extracting bases with high reading reliability of the sequencing among the bases of the sites detected by the comparison of the read sequences with the reference sequence in the step (3″).
[43] The method according to any one of [39] to [42], wherein the base length of the insertion or deletion site to be detected is preferably 10 bp or less, more preferably 1 to 5 bp.
[44] The method according to any one of [39] to [43] preferably, further comprising:
(7″) determining respective mutation frequencies for each of sites of the insertion or deletion mutations with different base lengths of the insertion or deletion and/or different types of the inserted or deleted bases in a control group by the same procedures as in the steps (1″) to (6″); and
(8″) subtracting the respective mutation frequencies in the control group obtained in the step (7″) from the respective mutation frequencies for each of the sites of the insertion or deletion mutations with different base lengths of the insertion or deletion and/or different types of the inserted or deleted bases in the test group obtained in the step (6″).
[45] The method according to [44], wherein preferably,

the method is a method for evaluating mutations in cancer cells, wherein the control group is a non-cancer cell population, or

the test group is a population of cells suspected of having cancer or cells to be evaluated for the risk of cancerization, the control group is a non-cancer cell population or a population of cells with a low risk of cancerization, and the risk of cancerization in the cells is evaluated by the method.

[46] The method according to [44], wherein preferably, the method is a method for evaluating genetic information in cultured cells, wherein the control group is a population of cells which are cultured cells of the same type as in the test group and have known genetic information.
[47] The method according to any one of [21] to [38], wherein preferably, the base pair substitution mutation is a one-base pair substitution mutation, a two-base pair substitution mutation, or a three-base pair substitution mutation.
[48] The method according to any one of [1] to [47], wherein a total amount of the read sequences used in the detection is preferably 1×10¹⁰ bp or less, more preferably 1×10⁹ bp or less, even more preferably 1×10⁸ bp or less, even more preferably 1×10⁷ bp or less, even more preferably 1×10⁶ bp or less.

### EXAMPLES

Hereinafter, the present invention will be described more specifically with reference to Examples.

### Example 1 Validation of Analysis Method

In this Example, the analysis method of the present invention was validated by analyzing synthetic DNA samples having known mutation frequencies by the test method of the present invention to qualitatively and quantitatively evaluate the mutations.

### 1. Preparation of DNA Sample

Synthetic DNA samples containing various mutation patterns in known amounts were prepared. Schematic diagram 1 shows a conceptual diagram of procedures of preparing the DNA samples. A synthetic DNA sequence having a 1000-bp random sequence (SEQ ID NO: 1; hereinafter, referred to as a random DNA sequence) was synthesized. This random DNA sequence contained approximately 50% each of GC and AT base pairs. On the basis of this random DNA sequence, DNA sequences harboring a mutation (base pair substitution mutation or short insertion or deletion mutation) (hereinafter, referred to as mutated DNA sequences) were prepared. Hereinafter, the details will be described.

For the mutated DNA sequences containing a base pair substitution mutation, 3 types of mutated sequences were prepared by substituting a GC base pair located at the center (position 501) of the random DNA sequence by a different base pair (GC→TA, CG or AT; see Table 1) and were each integrated into a pTAKN-2 vector. The obtained vectors were each dissolved in a TE buffer (pH 8.0, manufactured by Wako Pure Chemical Industries, Ltd.) and adjusted to a concentration of 100 ng/μL. The solutions containing the 3 types of mutated DNA sequences were mixed in equal amounts. Likewise, 3 types of mutated sequences were prepared by substituting an AT base pair (position 502) by a different base pair (AT→TA, CG or GC; see Table 1) and each integrated into a pTAKN-2 vector. TE buffer solutions (100 ng/μL) of the vectors were prepared, and 3 types of solutions thus obtained were mixed in equal amounts. Each mixed solution of equal amounts was used as a mutated DNA solution. Also, 10 μL of the mutated DNA solution was mixed with 90 μL of a TE buffer to prepare a 10-fold diluted mutated DNA solution. Further, 10 μL of the 10-fold diluted mutated DNA solution was mixed with 90 μL of a TE buffer to prepare a 100-fold diluted mutated DNA solution. Aside from these, the random DNA sequence was integrated into a pTAKN-2 vector, and a TE buffer solution (100 ng/μL) of the vector was prepared (random DNA solution). The random DNA solution was mixed with the mutated DNA solution, the 10-fold diluted mutated DNA solution, or the 100-fold diluted mutated DNA solution to prepare a DNA sample in which the substitutions of each base pair were found with equal frequencies and the total mutation frequency was 1/10³, 1/10⁴, 1/10⁵, or 1/10⁶ bp (see Table 2).

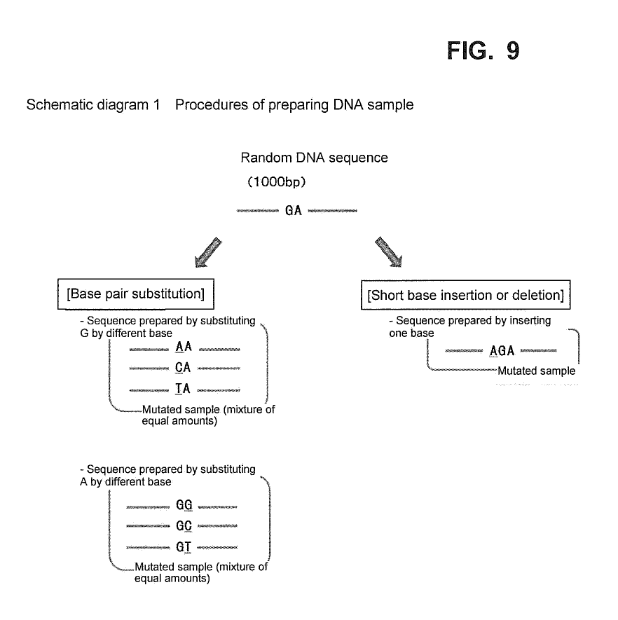

For the mutated DNA sequences containing a short insertion or deletion mutation, a mutated sequence was prepared by inserting one base (A, i.e., AT base pair) before the base pair at position 501 (see Table 1). In the same way as above, this sequence was integrated into a pTAKN-2 vector, and a TE buffer solution (100 ng/μL) of the vector was prepared and used as a mutated DNA solution. Also, 10-fold and 100-fold diluted mutated DNA solutions of the mutated sample solution were prepared. The random DNA solution was mixed with the mutated DNA solution, the 10-fold diluted mutated DNA solution, or the 100-fold diluted mutated DNA solution to prepare a DNA sample in which the total mutation frequency was 1/10³, 1/10⁴, 1/10⁵, or 1/10⁶ bp (see Table 2). The DNA sample containing each mutated DNA solution was used as a mutated sample, while a DNA sample free from the mutated DNA solution (consisting of the random DNA solution alone) was used as a control sample to conduct the following sequencing. Schematic diagram 1, entitled “Procedures of preparing DNA sample,” is shown on FIG. 9.

TABLE 1
1. Base pair substitution mutation

| | | | Transition |
|---|---|---|---|
| | Transversion | | (purine⇔purine or |
| | (purine⇔pyrimidine) | | pyrimidine⇔pyrimidine) |
| GC | GC > TA | GC > CG | GC > AT |
| AT | AT > TA | AT > CG | AT > GC |

2. Short insertion or deletion mutation
TTCTGAT > TTCT-A-GAT

TABLE 2
Ratio of mutated DNA solution and total mutation frequency
in DNA sample

| | Total mutation frequency (/bp) | | | | |
|---|---|---|---|---|---|
| | 1/10³ | 1/10⁴ | 1/10⁵ | 1/10⁶ | 0 |
| Amount of random DNA solution | — | 81 μL | 89 μL | 90 μL | 90 μL |
| Amount of mutated DNA solution | 90 μL | 9 μL | — | — | — |
| (base pair substitution mutation or | | | | | |
| short insertion or deletion mutation) | | | | | |
| Amount of 10-fold diluted mutated | — | — | 9 μL | — | — |
| DNA solution | | | | | |
| Amount of 100-fold diluted mutated | — | — | — | 9 μL | — |
| DNA solution | | | | | |
| Ratio of mutated DNA solution | 100% | 10% | 1% | 0.1% | 0% |

### 2. High-Throughput Sequencing

Each mutated sample and control sample prepared in the paragraph 1. were sequenced using a next-generation sequencer HiSeq 2500 (manufactured by, Illumina, Inc.; hereinafter, also referred to as HiSeq) according to the standard protocol. In this operation, DNA was fragmented into an average length of approximately 150 bp by ultrasonic treatment. Adaptors were added to both ends of each fragment. The fragments were sequenced at a read length of 2×125 bp. 1.9-Gbp nucleotide sequence information per sample was obtained on average.

### 3. Editing of Read Sequence and Preparation of Format for Mutation Analysis

Schematic diagram 2 shows a conceptual diagram of the editing and analysis flows of the read sequences obtained by the sequencing. First, the removal of the adaptor sequences at the ends of each read and the removal of bases with low quality were conducted using Cutadapt software (Martin, 2011).

i) In HiSeq, per DNA fragment (original fragment) subjected to the sequencing, a read 1 sequence is read from one side, and then, a read 2 sequence to be paired therewith is obtained from the opposite side of the fragment. Accordingly, each pair of read sequences selected by Cutadapt was constructed into one conjugated read using PEAR software by merging both the read sequences with each other at a part where the sequences are equivalent. All the bases in the sequence of the conjugated read were standardized to read 1. As the quality value of each base in the conjugated read, the sum of the quality values of the bases of read 1 and read 2 was adopted when the bases of read 1 and read 2 were complementary, whereas a value obtained by subtracting a smaller quality value from a larger quality value was adopted when the bases of read 1 and read 2 were not complementary. This procedure allows to select among the bases in the conjugated reads, bases of base pairs complementary between reads 1 and 2 according to the difference in quality value.

ii) The conjugated read of each prepared original fragment was mapped to a reference sequence (pTAKN-2 vector sequence having an insert of the random DNA sequence) using Bowtie 2 software to create a file of Sam format.

iii) The obtained Sam file was converted to a pileup format using Samtools software. In this operation, on the basis of the quality values of the bases, the scope of base information to be analyzed was restricted to the bases of the base pairs complementary between both reads in the merged regions of the pair reads.

iv) The obtained pileup format was subjected to mutation analysis using a program created using a programming language Python.

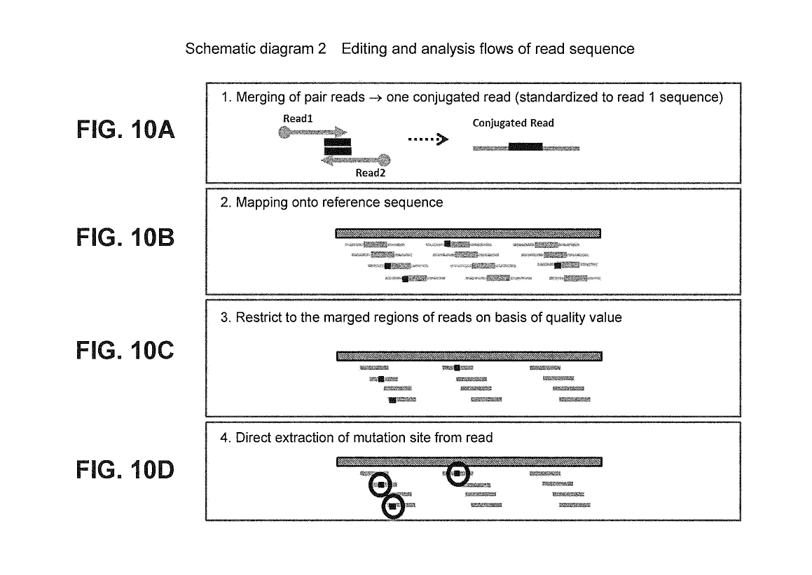

Schematic diagram 2, entitled “Editing and analysis flows of read sequence,” is shown on FIG. 10A-FIG. 10D.

### 4. Mutation Analysis

### 1) Detection of Base Pair Substitution Mutation

Schematic diagram 3 shows a conceptual diagram of a mutation analysis algorithm for the one-base pair substitution mutation. From the pileup format subjected to the analysis, all the bases to be analyzed in the read sequences were classified, using a program created using a programming language Python, into 4 groups: a group in which the corresponding base in the reference sequence was A; a group in which the corresponding base in the reference sequence was T; a group in which the corresponding base in the reference sequence was G; and a group in which the corresponding base in the reference sequence was C. Subsequently, the total number of bases assigned to each group and mutated bases were detected. From the obtained data, the mutation call ratio of each mutation pattern of an AT base pair (AT→TA, AT→CG, and AT→GC) to 10⁶ bp of the AT base pair before the mutation and the mutation call ratio of each mutation pattern of a GC base pair (GC→TA, GC→CG, and GC→AT) to 10⁶ bp of the GC base pair before the mutation were calculated.

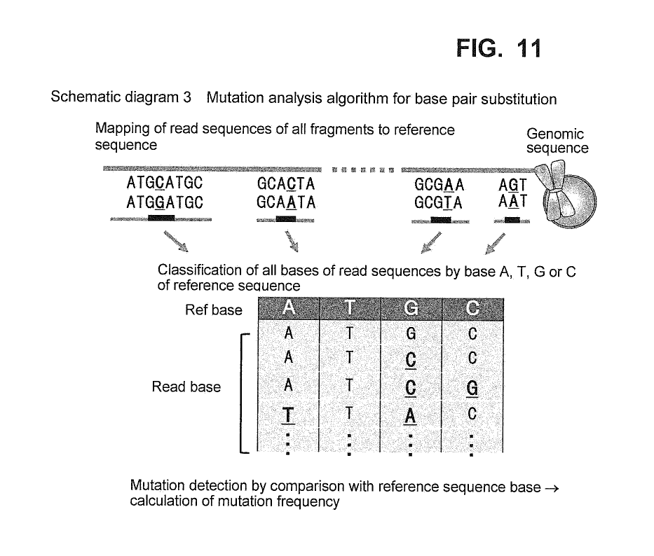

Schematic diagram 3, entitled “Mutation analysis algorithm for base pair substitution” is shown as FIG. 11.

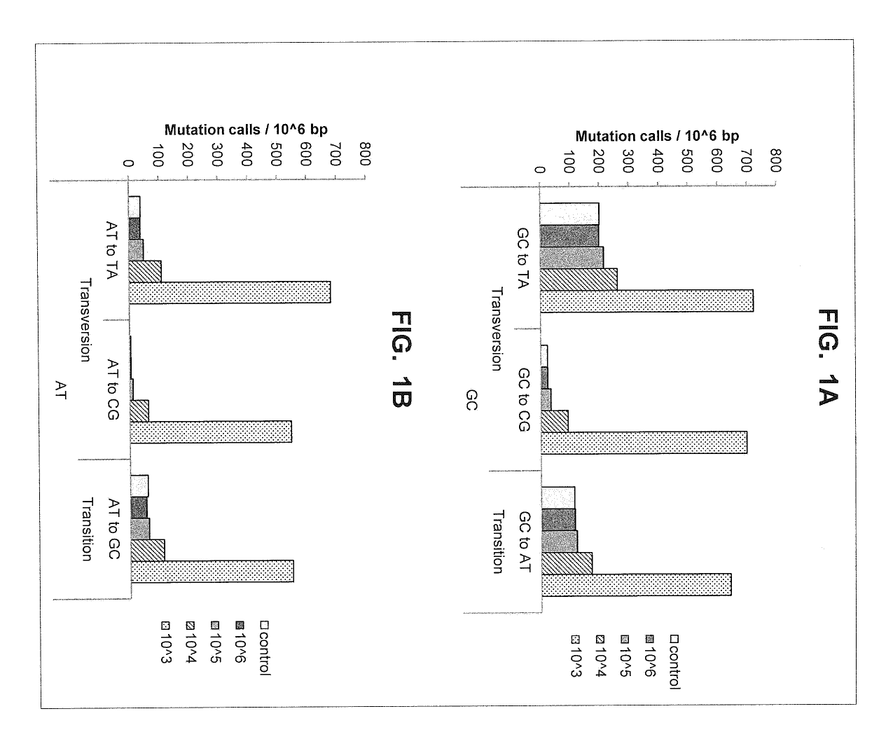

FIG. 1A and FIG. 1B show the respective mutation call ratio of the mutation patterns of each of GC and AT base pairs in the mutated samples containing a base pair substitution mutation. For all the mutation patterns, the mutation call ratios were elevated depending on the mutation frequencies in the samples. Mutations were also detected in the control samples, but which indicate background errors (errors generated in the process from sample preparation to sequencing, including a sequencing error). Furthermore, the mutation call ratios also differed among the mutation patterns in the control samples, and the GC base pair tended to exhibit a higher mutation call ratio as compared with the AT base pair. This is probably because the GC base pair is susceptible to chemical modification such as oxidation, in the process of library preparation such as DNA extraction.

### 2) Calculation of Increased Mutation Frequency

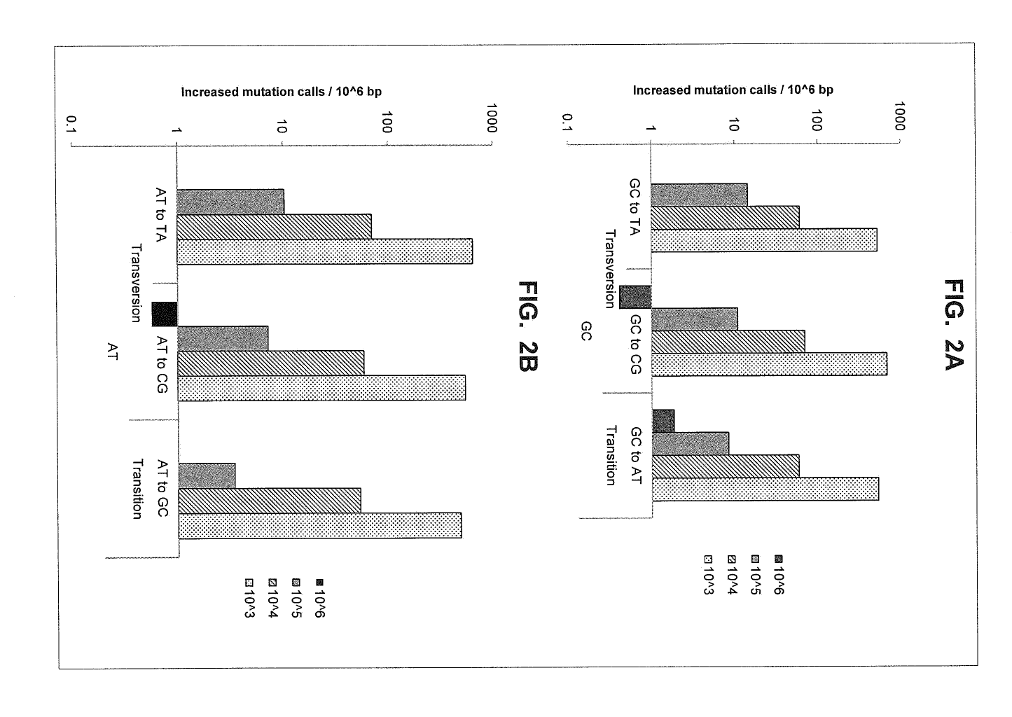

Next, the background errors including a sequencing error were excluded by subtracting the mutation call ratios of the control samples from the mutation call ratios of the mutated samples to calculate the amounts of mutation frequencies increased in the mutated samples with respect to the control samples (hereinafter, referred to as increased mutation frequencies). The calculated increased mutation frequencies are shown in FIG. 2A and FIG. 2B. For all the mutation patterns, the increased mutation frequencies were roughly consistent with the frequencies of the introduced mutations, demonstrating that a base pair substitution mutation with a frequency of one in every approximately 10⁵ bp was able to be detected by the method of the present invention.

### 3) Detection of Short Insertion or Deletion Mutation

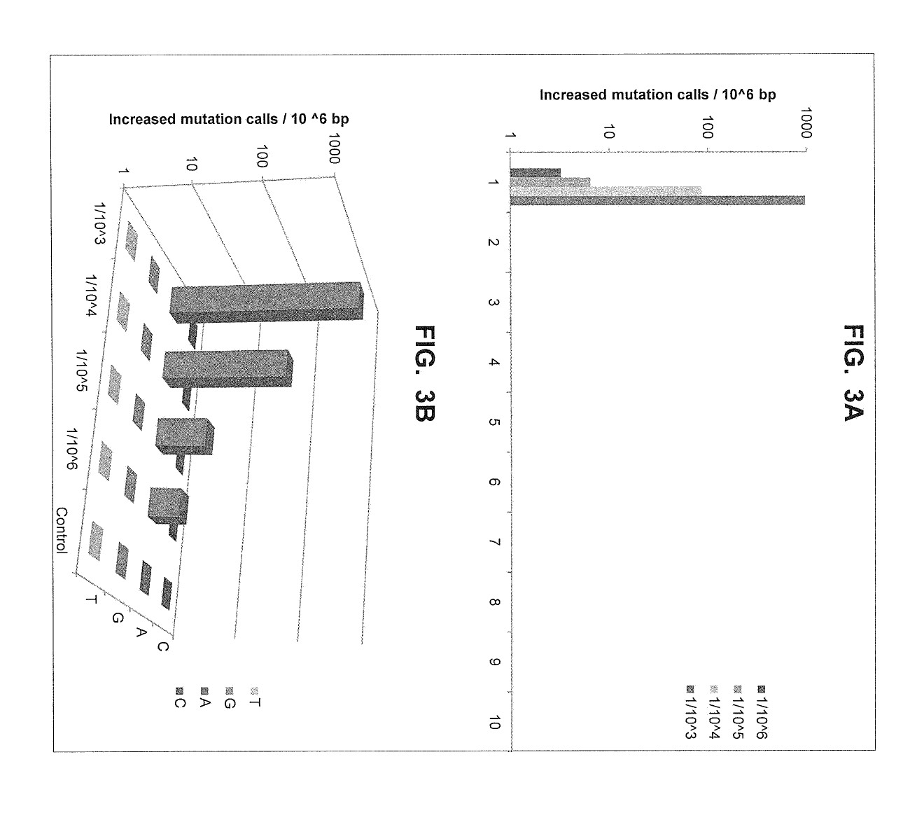

For the short insertion or deletion mutation, all the bases inserted or deleted at a length of 10 bp or less to the random DNA sequence were detected, and an appearance frequency was measured for each of the lengths of the insertions or deletions (bp) and each of the types of the inserted or deleted bases using a program created using a programming language Python. In the same way as in the base pair substitution mutation analysis mentioned above, background errors were excluded by subtracting the mutation frequencies of the control sample to calculate increased mutation frequencies. The results about the increased mutation frequencies of the insertion mutation are shown in FIG. 3A and FIG. 3B. FIG. 3A shows increased mutation frequencies as to the length of the inserted base (bp), and FIG. 3B shows increased mutation frequencies as to the type of the inserted base. A short insertion mutation with a frequency of one in every approximately 10⁵ bp was able to be detected by the method of the present invention. Also, a one-base deletion was studied in the same way as in the insertion mutation. As a result, a short deletion mutation with a frequency of one in every approximately 10⁵ bp was able to be detected (data not shown).

### 5. Conclusion

In this Example, comprehensive mutation information including quantity (frequency) and quality (mutation pattern) was able to be obtained highly sensitively as to a base pair substitution mutation and a short insertion or deletion mutation in DNA containing various pieces of mutation information. These results demonstrated that a low-frequency mutation present in DNA can be analyzed qualitatively and quantitatively by the analysis method of the present invention.

### Example 2 Analysis of Genotoxicity of Mutagen

In this Example, the genotoxicity of a mutagen was analyzed by qualitatively and quantitatively analyzing mutation patterns in the genome of an organism exposed to the mutagen according to the analysis method of the present invention. The mutagen used was ethylnitrosourea (ENU, CAS No. 759-73-9). A Salmonella TA100 strain which is available for detection of a base pair substitution mutation and is routinely used in the Ames test was used as the organism to be exposed to the mutagen. The experiment was conducted in 3 independent operations to prepare samples (n=3).

### 1. Exposure of TA100 Strain to Mutagen

The exposure to the mutagen was carried out in conformity to the preincubation method of the Ames test (K. Mortelmans et al., Mutat. Res. —Fundam. Mol. Mech. Mutagen., 2000, 455, 29-60). TA100 strain was inoculated to 2 mL of Nutrient Broth No. 2 (manufactured by Oxoid Ltd.) and shake-cultured at 37° C. at 180 rpm for 4 hours to obtain a preculture solution having an O.D. 660 value of 1.0 or more. ENU (54%; manufactured by Sigma-Aldrich Co. LLC) was diluted with dimethyl sulfoxide (DMSO; manufactured by Wako Pure Chemical Industries, Ltd.). One hundred μL of the ENU solution diluted into an appropriate concentration, 500 μL of a 0.1 M phosphate buffer, and 100 μL of the preculture solution were added into a test tube (ENU concentration: 67.5, 135, 270, 405, 540, 810, and 1080 μg/tube), and shake-cultured at 100 rpm for 20 minutes in a water bath of 37° C. For a control group, 100 μL of a solvent (DMSO) was added instead of the ENU solution. After the shake culture for 20 minutes, the test tube containing the culture solution was taken out of the water bath. Fifty μL of the culture solution was added to 2 mL of a nutrient broth solution dispensed in advance, and additionally cultured at 37° C. at 180 rpm for 14 hours. Then, 1 mL of the bacterial suspension was recovered and centrifuged at 7,500 rpm for 5 minutes. The supernatant was removed to recover bacterial cells.

For the Ames test, a bacterial suspension exposed to ENU under the same conditions as above was also prepared. Two mL of top agar (containing 1% NaCl, 1% agar, 0.05 mM histidine and 0.05 mM biotin) warmed to 45° C. was added thereto, and the bacterial cells were suspended by vortex. Then, the suspension was layered over a minimal glucose agar medium (Tesmedia (Registered trademark) AN; manufactured by Oriental Yeast Co., Ltd.). The obtained plate was cultured at 37° C. for 48 hours. Then, observed colonies were counted.

### 2. Recovery of Total DNA

From the bacterial cells obtained in the paragraph 1., total DNA was recovered using DNeasy Blood & Tissue Kit (manufactured by Qiagen N.V.) according to the recommended protocol.

### 3. High-Throughput Sequencing

The total DNA solutions recovered in the paragraph 2. from the control group and the ENU treatment groups were sequenced using a next-generation sequencer HiSeq 2500 (manufactured by Illumina, Inc.) according to the standard protocol. In this operation, DNA was fragmented into an average length of approximately 150 bp by ultrasonic treatment. Adaptors were added to both ends of each fragment. The fragments were sequenced at a read length of 2×125 bp. 5.0-Gbp nucleotide sequence information per sample was obtained on average.

### 4. Editing of Read Sequence and Mutation Analysis

The editing of read sequences obtained by the sequencing and mutation analysis were conducted using Cutadapt software, PEAR software, Bowtie 2 software, Samtools software, and a program created using a programming language Python according to the conceptual diagram of analysis flow shown in Schematic diagram 2 in the same way as in Example 1. In this Example, PCR duplicates were removed using Picard tools (broadinstitute.github.io/picard/) after mapping using the Bowtie 2 software.

A reference sequence for the mapping using the Bowtie 2 software was constructed on the basis of the genomic sequence of the S. typhimurium TA100 strain. First, DNA was extracted from the TA100 strain and sequenced using a next-generation sequencer HiSeq 2500 (manufactured by Illumina, Inc.) according to the standard protocol. In this operation, the DNA was fragmented into an average length of approximately 300 bp by ultrasonic treatment. Adaptors were added to both ends of each fragment. The fragments were sequenced at a read length of 2×125 bp. The obtained read sequences were mapped to the genomic sequence of a S. typhimurium LT-2 strain, a pSLT plasmid sequence (GCA000006945.2), and a R46 plasmid sequence (NC_003292.1), followed by mutation detection using Samtools software. A reference sequence based on the thus-obtained genomic sequence of the TA100 strain reflecting the mutation information was prepared and used in the mutation analysis. The genomic sequence of the TA100 strain is shown in SEQ ID NO: 2.

### 5. Calculation of Increased Mutation Frequency

By the same procedures as in Example 1, all the bases to be analyzed in all the read sequences mapped to the reference sequence were assigned, using a program created using a programming language Python, to 4 groups according to corresponding bases in the reference sequence. Subsequently, the total number of bases in each group and mutated bases with respect to the reference sequence were detected. Each mutation pattern (AT→TA, AT→CG, and AT→GC; and GC→TA, GC→CG, and GC→AT) in 10⁶ bp of the AT base pair or the GC base pair in the bases to be analyzed, and the mutation call ratio of each mutation pattern were calculated as to each of the ENU treatment groups and the control group by comparing the mutated bases with the bases of the reference sequence. Statistical study of the frequency of each mutation pattern between the control group and the ENU treatment groups was conducted for each mutation pattern by the Dunnett's multiple comparison test.

Subsequently, increased mutation frequencies due to exposure to ENU were calculated by subtracting the mutation call ratio of the control group from the mutation call ratio of the ENU treatment group for each base pair mutation pattern by the same procedures as in the paragraph 2) of Example 1.

### 6. Sequence Context Analysis

In this study, mutation call ratios were analyzed on the basis of a sequence context. Specifically, the mutation types of base pairs at the respective locations of the mutation calls were detected on the basis of the mutation call information and the reference sequence information obtained in the paragraph 5. Further, information on 3 bases including a base at the location of each mutation call ant both of its adjacent bases was collected on the basis of the reference sequence information. The mutation at the location of each mutation call was classified into 96 types according to the 6 base pair mutation types and information on both the adjacent bases (4×4=16). A mutation call ratio (/10⁶ bp) was calculated for each of the 96 mutation types based on this sequence context. Increased mutation frequencies due to exposure to ENU were calculated by subtracting the mutation call ratio of the control group from the mutation call ratio of the ENU treatment group for each mutation type.

### 7. Results

### 1) The Number of Revertants of Ames Test

Table 3 shows the number of revertant colonies after exposure to ENU. The data was indicated by a mean and standard deviation of measurement values from 3 plates. Increase in the number of revertants was found by the exposure to ENU, demonstrating that mutations were introduced into the genome of the TA100 strain by the exposure to ENU.

TABLE 3

| ENU | |
|---|---|
| concentration | The number |
| (μg/plate) | of revertants |
| 0 (DMSO) | 110 ± 10.0 |
| 67.5 | 258 ± 2.5 |
| 135 | 659 ± 113.9 |
| 270 | 3320 ± 734.4 |
| 405 | 4994 ± 455.6 |
| 540 | 5051 ± 173.0 |
| 810* | 3102 ± 883.0 |
| 1080* | 503 ± 190.9 |

*Growth inhibition of the strain was found on plate.

### 2) Calculation of Change in Mutation Call Ratio by Exposure to ENU

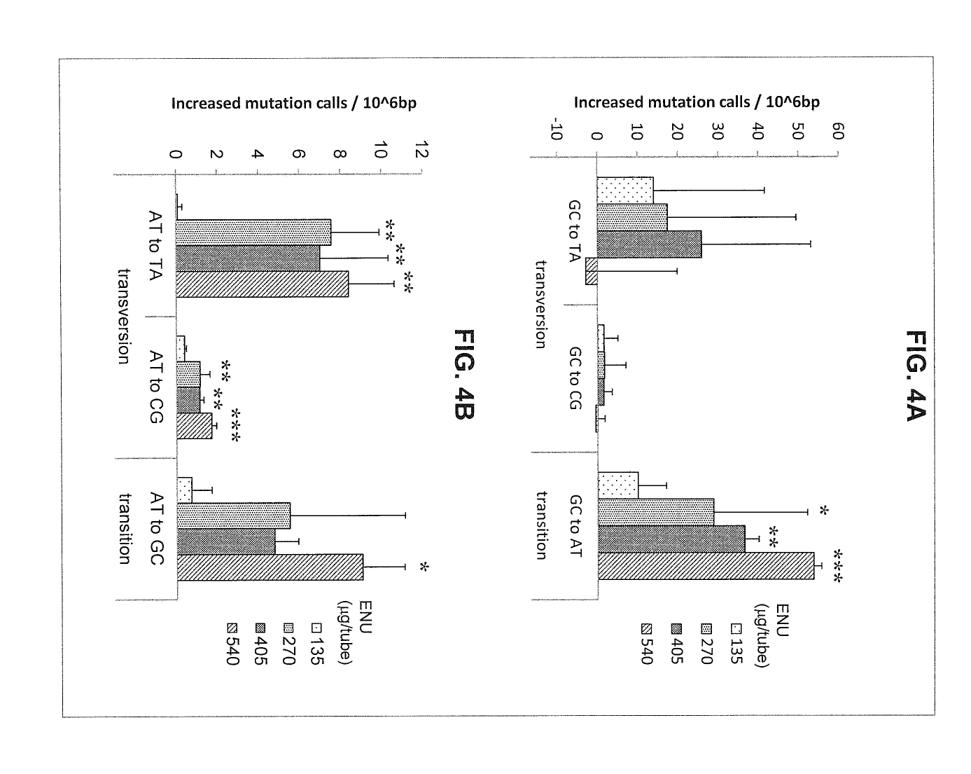

FIG. 4A and FIG. 4B show the increased mutation frequencies calculated in the paragraph 5. as to the ENU treatment groups (ENU concentration: 135, 270, 405 and 540 μg/tube). Increase was found in the frequencies of a plurality of base pair mutation patterns by the exposure to ENU. For the GC base pair, increase in the frequency of GC>AT mutation was found (FIG. 4A). On the other hand, for the AT base pair, increase in the frequencies of AT>TA and AT>GC mutations was mainly found (FIG. 4B).

### 3) Classification of Each Mutation Pattern According to Original Base

In HiSeq, the sequence of read 1 corresponds to the original DNA fragment (original fragment) subjected to sequencing reaction. Thus, examining bases among A, T, G and C in the reference sequence on which the bases of read 1 sequences are mapped leads to elucidate a base before the mutation (i.e., an original base) at the location of a mutation in the original fragment.

Since a background error frequency can differ depending on original bases, the mutation patterns were further classified according to original bases, and the increased mutation frequency of each class was determined. Specifically, each of the 6 base pair mutation patterns in the control sample and the mutated samples determined in the paragraph 5. was further classified into 2 groups according to the types of the original bases, and the mutation frequency of each class was determined. Subsequently, increased mutation frequencies were calculated by subtracting the corresponding mutation frequency of the control sample from the mutation frequency of the mutated sample.

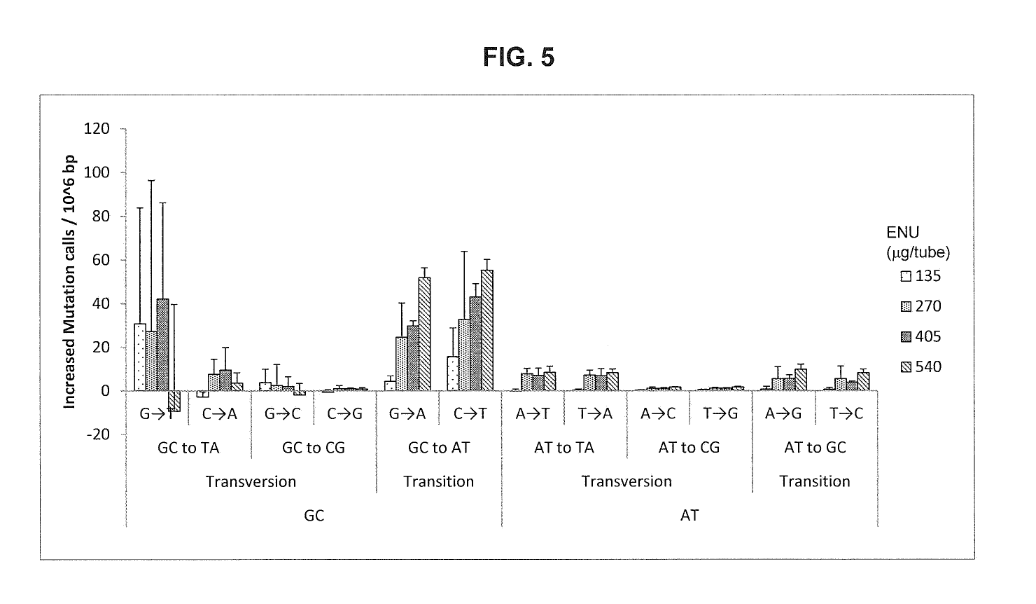

FIG. 5 shows the increased mutation frequencies of the base pair mutation patterns divided according to the types of the original bases in the ENU-exposed samples. Normally, increase in mutation frequency is equivalent in both bases constituting the base pair since both bases of a base pair were altered in the mutation fixed in the genome by exposure to ENU. However, in the GC>TA mutation shown in FIG. 5, the tendency of an evidently higher mutation frequency was found for the original base G as compared with the original base C.

The bias described above strongly suggests that a great majority of GC>TA mutations were caused by chemically modified G. It was thus considered that increase in the frequency of GC>TA mutation found in this study reflected a reading error of a chemically modified G base in sequencing, not the mutation fixed in the genome. It is known that the G base is susceptible to chemical modification by oxidation in the process of DNA sample preparation and is prone to errors to misread G as T in sequencing. Thus, the increase in GC>TA mutation frequency found in some samples in this study seemed to be artificial influence due to the oxidation of G in the process of DNA preparation. These results suggested that a sequencing error caused by an error in the process of DNA sample preparation from cells can be removed by the analysis method of the present invention.

### 4) Spectral Analysis of Mutation

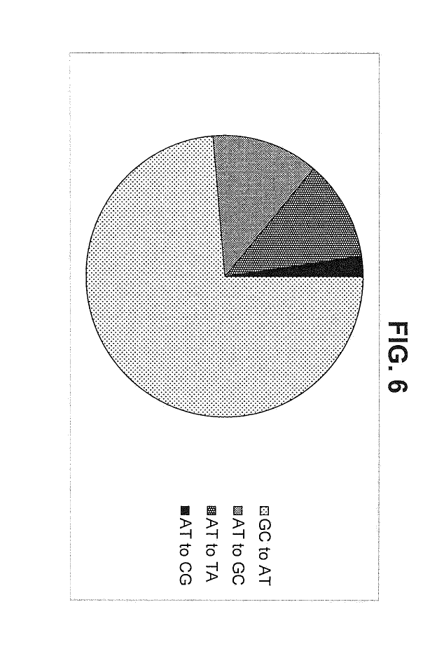

Next, mutation spectra were analyzed on the basis of the increased mutation frequency of each mutation pattern found to be increased in the ENU 540 μg/tube group. Specifically, the increased mutation frequencies of the mutation patterns in 10⁶ bp were summed, and the ratio of each mutation pattern to the whole was calculated. The results are shown in FIG. 6 and Table 4. A mutation pattern found to have the largest increase in frequency was GC>AT mutation, and a mutation pattern having the second highest ratio was AT>GC mutation. This was similar to results about mutation patterns induced by ENU found in a YG7108 strain, a bacterial strain of the Ames test, in Non Patent Literature 5, suggesting that mutation patterns induced by ENU were able to be accurately detected by the method of the present invention.

TABLE 4

| Base pair | |
|---|---|
| substitution | % |
| GC > AT | 73.7 |
| AT > GC | 12.4 |
| AT > TA | 11.5 |
| AT > CG | 2.36 |

### 5) Sequence Context Analysis

FIG. 7 and FIG. 8 show increased mutation frequencies calculated by sequence context analysis. Each mutation pattern in the drawings is indicated by the mutation pattern of a pyrimidine base (C>A, C>G, C>T, T>A, T>C and T>G) among mutated base pairs, and a 3-base sequence including the pyrimidine base and both of its adjacent bases (e.g., the C>T wherein the C base flanked by A and T is indicated by ACT). In this analysis, for C>T mutation, which has the largest increase in mutation frequency, the tendency of larger increase in mutation frequency was found in a context with a pyrimidine base (C or T) located at 3′ side to the C. This was similar to the patterns shown in the mutation signature by an alkylating agent (Signature 11 in FIG. 7 and FIG. 8; see Nature, 2013, 500 (7463): 415-421). These results were not contradictory to the fact that ENU is an alkylating agent. These results suggest that sequence context analysis is a useful method which can predict a mechanism underlying the genotoxicity of a mutagen or conveniently predict a role of the mutagen in carcinogenesis in humans.

1. A method for evaluating the genotoxicity of a test substance, comprising:
(1) obtaining DNAs from a test group, the test group is a cell population exposed to the test substance;
(2) sequencing fragments of the DNAs to obtain one or more read sequences per fragment;
(3) comparing each of the one or more read sequences with a reference sequence to detect sites of mismatch bases between the each read sequence and the reference sequence, wherein the reference sequence is a known sequence in the DNAs;
(4) obtaining the sites detected in the step (3) as sites of mutation each having a base pair substitution mutation;
(5) classifying each of the obtained mutations according to base pair mutation patterns; and
(6) determining respective mutation frequencies of the mutation patterns obtained in the step (5).

2. The method according to claim 1, further comprising extracting, from the bases of the read sequences obtained in the step (2), bases with high reading reliability of the sequencing, wherein
the step (3) comprises comparing the extracted bases on the read sequences with the bases of the reference sequence.

3. The method according to claim 1, wherein
the steps (3) to (5) comprise:
dividing bases contained in the read sequences into the following bases (i) to (iv):
(i) a base located at a position where the base on the reference sequence is A,
(ii) a base located at a position where the base on the reference sequence is T,
(iii) a base located at a position where the base on the reference sequence is G, and
(iv) a base located at a position where the base on the reference sequence is C;
detecting mismatch bases with respect to the reference sequence from among the bases contained in the read sequences, and obtaining sites of the detected bases as sites of mutation each having a base pair substitution mutation;
obtaining base pairs before mutation and after mutation at the respective sites of mutation of the detected mismatch bases; and
classifying the base pair substitution mutations at the sites of mutation, according to the types of the base pairs before mutation and the base pairs after mutation, into 6 of base pair mutation patterns: AT→TA, AT→CG, AT→GC, GC→TA, GC→CG, and GC→AT.

4. The method according to claim 1, further comprising:
(7) conducting the same procedures as in the steps (1) to (6) on a control group, the control group is a cell population unexposed to the test substance, thereby determining respective mutation frequencies of the base pair mutation patterns in the control group; and
(8) subtracting the respective mutation frequencies of the mutation patterns in the control group obtained in the step (7) from the respective mutation frequencies of the mutation patterns in the test group obtained in the step (6).

5. A method for evaluating the genotoxicity of a test substance, comprising:
(1′) obtaining DNAs from a test group, the test group is a cell population exposed to the test substance;
(2′) sequencing fragments of the DNAs to obtain one or more read sequences per fragment;
(3′) comparing each of the one or more read sequences with a reference sequence to detect sites of mismatch bases between the each read sequence and the reference sequence, wherein the reference sequence is a known sequence in the DNAs;
(4′) obtaining the sites detected in the step (3′) as sites of mutation each having a base pair substitution mutation;
(5′) as to each of the mutations thus obtained, determining a context sequence comprising a base before mutation and adjacent upstream and downstream bases of the base before mutation, the context sequence is determined on the basis of the reference sequence;
(6′) typing each of the mutations obtained in the step (4′) according to the context sequence determined in the step (5′) and the type of the base after mutation; and
(7′) determining respective mutation frequencies of the mutation types obtained in the step (6′).

6. The method according to claim 5, further comprising extracting, from the bases of the read sequences obtained in the step (2′), bases with high reading reliability of the sequencing, wherein
the step (3′) comprises comparing the extracted bases on the read sequences with the bases of the reference sequence.

7. The method according to claim 5, wherein the steps (3′) to (6′) comprise:
dividing bases contained in the read sequences into the following bases (i) to (iv):
(i) a base located at a position where the base on the reference sequence is A,
(ii) a base located at a position where the base on the reference sequence is T,
(iii) a base located at a position where the base on the reference sequence is G, and
(iv) a base located at a position where the base on the reference sequence is C;
detecting mismatch bases with respect to the reference sequence from among the bases contained in the read sequences, and obtaining sites of the detected bases as sites of mutation each having a base pair substitution mutation;
obtaining base pairs before mutation and after mutation at the respective sites of mutation of the detected mismatch bases;
classifying the base pair substitution mutations at the sites of mutation, according to the types of the base pairs before mutation and the base pairs after mutation, into 6 of base pair mutation patterns: AT→TA, AT→CG, AT→GC, GC→TA, GC→CG, and GC→AT;
determining context sequences each consisting of a base before mutation located at each of the sites of mutation, one or more adjacent upstream bases of the base before mutation, and one or more adjacent downstream bases of the base before mutation; and
typing each of the base pair substitution mutations according to the 6 of base pair mutation patterns and the context sequences.

8. The method according to claim 5, further comprising:
(8′) conducting the same procedures as in the steps (1′) to (7′) on a control group, the control group is a cell population unexposed to the test substance, thereby determining respective mutation frequencies of the mutation types in the control group; and
(9′) subtracting the respective mutation frequencies of the mutation types in the control group obtained in the step (8′) from the respective mutation frequencies of the mutation types in the test group obtained in the step (7′).

9. A method for evaluating the genotoxicity of a test substance, comprising:
(1″) obtaining DNAs from a test group, the test group is a cell population exposed to the test substance;
(2″) sequencing fragments of the DNAs to obtain one or more read sequences per fragment;
(3″) comparing each of the one or more read sequences with a reference sequence to detect sites of base insertion or deletion in the each read sequence with respect to the reference sequence, wherein the reference sequence is a known sequence in the DNAs;
(4″) obtaining the sites detected in the step (3″) as sites of mutation each having an insertion or deletion mutation;
(5″) as to each of the mutations thus obtained, determining the base length of the insertion or deletion and/or the type of the inserted or deleted base; and
(6″) determining respective mutation frequencies for each of the sites of the insertion or deletion mutations with different base lengths of the insertion or deletion and/or different types of the inserted or deleted bases determined in the step (5″).

10. The method according to claim 9, further comprising extracting, from the bases of the read sequences obtained in the step (2″), bases with high reading reliability of the sequencing, wherein
the step (3″) comprises comparing the extracted bases on the read sequences with the bases of the reference sequence.

11. The method according to claim 9, further comprising:
(7″) conducting the same procedures as in the steps (1″) to (6″) on a control group, the control group is a cell population unexposed to the test substance, thereby determining respective mutation frequencies for each of the sites of the insertion or deletion mutations with different base lengths of the insertion or deletion and/or different types of the inserted or deleted bases in the control group; and
(8″) subtracting the respective mutation frequencies in the control group obtained in the step (7″) from the respective mutation frequencies for each of the sites of the insertion or deletion mutations with different base lengths of the insertion or deletion and/or different types of the inserted or deleted bases in the test group obtained in the step (6″).

12. A method for evaluating mutations in cancer cells, comprising:
(1) obtaining DNAs from a test group, the test group is a cancer cell population;
(2) sequencing fragments of the DNAs to obtain one or more read sequences per fragment;
(3) comparing each of the one or more read sequences with a reference sequence to detect sites of mismatch bases between the each read sequence and the reference sequence, wherein the reference sequence is a known sequence in the DNAs;
(4) obtaining the sites detected in the step (3) as sites of mutation each having a base pair substitution mutation;
(5) classifying each of the obtained mutations according to base pair mutation patterns; and
(6) determining respective mutation frequencies of the mutation patterns obtained in the step (5).

13. A method for evaluating genetic information in cultured cells, comprising:
(1) obtaining DNAs from a test group, the test group is a cultured cell population;
(2) sequencing fragments of the DNAs to obtain one or more read sequences per fragment;
(3) comparing each of the one or more read sequences with a reference sequence to detect sites of mismatch bases between the each read sequence and the reference sequence, wherein the reference sequence is a known sequence in the DNAs;
(4) obtaining the sites detected in the step (3) as sites of mutation each having a base pair substitution mutation;
(5) classifying each of the obtained mutations according to base pair mutation patterns; and
(6) determining respective mutation frequencies of the mutation patterns obtained in the step (5).

14. The method according to claim 12, further comprising extracting, from the bases of the read sequences obtained in the step (2), bases with high reading reliability of the sequencing, wherein
the step (3) comprises comparing the extracted bases on the read sequences with the bases of the reference sequence.

15. The method according to claim 13, further comprising extracting, from the bases of the read sequences obtained in the step (2), bases with high reading reliability of the sequencing, wherein
the step (3) comprises comparing the extracted bases on the read sequences with the bases of the reference sequence.

16. The method according to claim 12, wherein the steps (3) to (5) comprise:
dividing bases contained in the read sequences into the following bases (i) to (iv):
(i) a base located at a position where the base on the reference sequence is A,
(ii) a base located at a position where the base on the reference sequence is T,
(iii) a base located at a position where the base on the reference sequence is G, and
(iv) a base located at a position where the base on the reference sequence is C;
detecting mismatch bases with respect to the reference sequence from among the bases contained in the read sequences, and obtaining sites of the detected bases as sites of mutation each having a base pair substitution mutation;
obtaining base pairs before mutation and after mutation at the respective sites of mutation of the detected mismatch bases; and
classifying the base pair substitution mutations at the sites of mutation, according to the types of the base pairs before mutation and the base pairs after mutation, into 6 of base pair mutation patterns: AT→TA, AT→CG, AT→GC, GC→TA, GC→CG, and GC→AT.

17. The method according to claim 13, wherein the steps (3) to (5) comprise:
dividing bases contained in the read sequences into the following bases (i) to (iv):
(i) a base located at a position where the base on the reference sequence is A,
(ii) a base located at a position where the base on the reference sequence is T,
(iii) a base located at a position where the base on the reference sequence is G, and
(iv) a base located at a position where the base on the reference sequence is C;
detecting mismatch bases with respect to the reference sequence from among the bases contained in the read sequences, and obtaining sites of the detected bases as sites of mutation each having a base pair substitution mutation;
obtaining base pairs before mutation and after mutation at the respective sites of mutation of the detected mismatch bases; and
classifying the base pair substitution mutations at the sites of mutation, according to the types of the base pairs before mutation and the base pairs after mutation, into 6 of base pair mutation patterns: AT→TA, AT→CG, AT→GC, GC→TA, GC→CG, and GC→AT.

18. The method according to claim 12, further comprising:
(7) determining respective mutation frequencies of base pair mutation patterns in a control group by the same procedures as in the steps (1) to (6); and
(8) subtracting the respective mutation frequencies of the mutation patterns in the control group obtained in the step (7) from the respective mutation frequencies of the mutation patterns in the test group obtained in the step (6).

19. The method according to claim 13, further comprising:
(7) determining respective mutation frequencies of base pair mutation patterns in a control group by the same procedures as in the steps (1) to (6); and
(8) subtracting the respective mutation frequencies of the mutation patterns in the control group obtained in the step (7) from the respective mutation frequencies of the mutation patterns in the test group obtained in the step (6).

20. A method for evaluating mutations in cancer cells, comprising:
(1′) obtaining DNAs from a test group, the test group is a cancer cell population;
(2′) sequencing fragments of the DNAs to obtain one or more read sequences per fragment;
(3′) comparing each of the one or more read sequences with a reference sequence to detect sites of mismatch bases between the each read sequence and the reference sequence, wherein the reference sequence is a known sequence in the DNAs;
(4′) obtaining the sites detected in the step (3′) as sites of mutation each having a base pair substitution mutation;
(5′) as to each of the mutations thus obtained, determining a context sequence comprising a base before mutation and adjacent upstream and downstream bases of the base before mutation, the context sequence is determined on the basis of the reference sequence;
(6′) typing each of the mutations obtained in the step (4′) according to the context sequence determined in the step (5′) and the type of the base after mutation; and
(7′) determining respective mutation frequencies of the mutation types obtained in the step (6′).

21. A method for evaluating genetic information in cultured cells, comprising:
(1′) obtaining DNAs from a test group, the test group is a cultured cell population;
(2′) sequencing fragments of the DNAs to obtain one or more read sequences per fragment;
(3′) comparing each of the one or more read sequences with a reference sequence to detect sites of mismatch bases between the each read sequence and the reference sequence, wherein the reference sequence is a known sequence in the DNAs;
(4′) obtaining the sites detected in the step (3′) as sites of mutation each having a base pair substitution mutation;
(5′) as to each of the mutations thus obtained, determining a context sequence comprising a base before mutation and adjacent upstream and downstream bases of the base before mutation, the context sequence is determined on the basis of the reference sequence;
(6′) typing each of the mutations obtained in the step (4′) according to the context sequence determined in the step (5′) and the type of the base after mutation; and
(7′) determining respective mutation frequencies of the mutation types obtained in the step (6′).

22. The method according to claim 20, further comprising extracting, from the bases of the read sequences obtained in the step (2′), bases with high reading reliability of the sequencing, wherein
the step (3′) comprises comparing the extracted bases on the read sequences with the bases of the reference sequence.

23. The method according to claim 21, further comprising extracting, from the bases of the read sequences obtained in the step (2′), bases with high reading reliability of the sequencing, wherein
the step (3′) comprises comparing the extracted bases on the read sequences with the bases of the reference sequence.

24. The method according to claim 20, wherein
the steps (3′) to (6′) comprise:
dividing bases contained in the read sequences into the following bases (i) to (iv):
(i) a base located at a position where the base on the reference sequence is A,
(ii) a base located at a position where the base on the reference sequence is T,
(iii) a base located at a position where the base on the reference sequence is G, and
(iv) a base located at a position where the base on the reference sequence is C;
detecting mismatch bases with respect to the reference sequence from among the bases contained in the read sequences, and obtaining sites of the detected bases as sites of mutation each having a base pair substitution mutation;
obtaining base pairs before mutation and after mutation at the respective sites of mutation of the detected base mismatch;
classifying the base pair substitution mutations at the sites of mutation, according to the types of the base pairs before mutation and the base pairs after mutation, into 6 of base pair mutation patterns: AT→TA, AT→CG, AT→GC, GC→TA, GC→CG, and GC→AT;
determining context sequences each consisting of a base before mutation located at each of the sites of mutation, one or more adjacent upstream bases of the base before mutation, and one or more adjacent downstream bases of the base before mutation; and
typing each of the base pair substitution mutations according to the 6 of base pair mutation patterns and the context sequences.

25. The method according to claim 21, wherein
the steps (3′) to (6′) comprise:
dividing bases contained in the read sequences into the following bases (i) to (iv):
(i) a base located at a position where the base on the reference sequence is A,
(ii) a base located at a position where the base on the reference sequence is T,
(iii) a base located at a position where the base on the reference sequence is G, and
(iv) a base located at a position where the base on the reference sequence is C;
detecting mismatch bases with respect to the reference sequence from among the bases contained in the read sequences, and obtaining sites of the detected bases as sites of mutation each having a base pair substitution mutation;
obtaining base pairs before mutation and after mutation at the respective sites of mutation of the detected base mismatch;
classifying the base pair substitution mutations at the sites of mutation, according to the types of the base pairs before mutation and the base pairs after mutation, into 6 of base pair mutation patterns: AT→TA, AT→CG, AT→GC, GC→TA, GC→CG, and GC→AT;
determining context sequences each consisting of a base before mutation located at each of the sites of mutation, one or more adjacent upstream bases of the base before mutation, and one or more adjacent downstream bases of the base before mutation; and
typing each of the base pair substitution mutations according to the 6 of base pair mutation patterns and the context sequences.

26. The method according to claim 20, further comprising:
(8′) determining respective mutation frequencies of mutation types in a control group by the same procedures as in the steps (1′) to (7′); and
(9′) subtracting the respective mutation frequencies of the mutation types in the control group obtained in the step (8′) from the respective mutation frequencies of the mutation types in the test group obtained in the step (7′).

27. The method according to claim 21, further comprising:
(8′) determining respective mutation frequencies of mutation types in a control group by the same procedures as in the steps (1′) to (7′); and
(9′) subtracting the respective mutation frequencies of the mutation types in the control group obtained in the step (8′) from the respective mutation frequencies of the mutation types in the test group obtained in the step (7′).

28. A method for evaluating mutations in cancer cells, comprising:
(1″) obtaining DNAs from a test group, the test group is a cancer cell population;
(2″) sequencing fragments of the DNAs to obtain one or more read sequences per fragment;
(3″) comparing each of the one or more read sequences with a reference sequence to detect sites of base insertion or deletion in the each read sequence with respect to the reference sequence, wherein the reference sequence is a known sequence in the DNAs;
(4″) obtaining the sites detected in the step (3″) as sites of mutation each having an insertion or deletion mutation;
(5″) as to each of the mutations thus obtained, determining the base length of the insertion or deletion and/or the type of the inserted or deleted base; and
(6″) determining respective mutation frequencies for each of the sites of the insertion or deletion mutations with different base lengths of the insertion or deletion and/or different types of the inserted or deleted bases determined in the step (5″).

29. A method for evaluating genetic information in cultured cells, comprising:
(1″) obtaining DNAs from a test group, the test group is a cultured cell population;
(2″) sequencing fragments of the DNAs to obtain one or more read sequences per fragment;
(3″) comparing each of the one or more read sequences with a reference sequence to detect sites of base insertion or deletion in the each read sequence with respect to the reference sequence, wherein the reference sequence is a known sequence in the DNAs;
(4″) obtaining the sites detected in the step (3″) as sites of mutation each having an insertion or deletion mutation;
(5″) as to each of the mutations thus obtained, determining the base length of the insertion or deletion and/or the type of the inserted or deleted base; and
(6″) determining respective mutation frequencies for each of the sites of the insertion or deletion mutations with different base lengths of the insertion or deletion and/or different types of the inserted or deleted bases determined in the step (5″).

30. The method according to claim 28, further comprising extracting, from the bases of the read sequences obtained in the step (2″), bases with high reading reliability of the sequencing, wherein
the step (3″) comprises comparing the extracted bases on the read sequences with the bases of the reference sequence.

31. The method according to claim 29, further comprising extracting, from the bases of the read sequences obtained in the step (2″), bases with high reading reliability of the sequencing, wherein
the step (3″) comprises comparing the extracted bases on the read sequences with the bases of the reference sequence.

32. The method according to claim 28, further comprising:
(7″) determining respective mutation frequencies for each of sites of the insertion or deletion mutations with different base lengths of the insertion or deletion and/or different types of the inserted or deleted bases in a control group by the same procedures as in the steps (1″) to (6″); and
(8″) subtracting the respective mutation frequencies in the control group obtained in the step (7″) from the respective mutation frequencies for each of the sites of the insertion or deletion mutations with different base lengths of the insertion or deletion and/or different types of the inserted or deleted bases in the test group obtained in the step (6″).

33. The method according to claim 29, further comprising:
(7″) determining respective mutation frequencies for each of sites of the insertion or deletion mutations with different base lengths of the insertion or deletion and/or different types of the inserted or deleted bases in a control group by the same procedures as in the steps (1″) to (6″); and
(8″) subtracting the respective mutation frequencies in the control group obtained in the step (7″) from the respective mutation frequencies for each of the sites of the insertion or deletion mutations with different base lengths of the insertion or deletion and/or different types of the inserted or deleted bases in the test group obtained in the step (6″).
